(19) United States
(12) Patent Application Publication
(10) Pub. No.: US 2026/0277199 A1
(43) Pub. Date: Sep. 17, 2026

(54) METHODS AND ARRANGEMENTS FOR INLINE, ONLINE, AT-LINE, AND OFFLINE PROCESS ANALYTICAL TECHNOLOGY TESTING (PAT) BOXES, FRACTION COLLECTORS, SKIDS AND AUTOMATED SAMPLING MODULES

(71) Applicant: Arranta Bio Holdings, LLC., Watertown, MA (US)

(72) Inventors: Aaron Blake Cowley, Watertown, MA (US); Mark Jandreski, Watertown, MA (US); Edita Botonjic-Sehic, Watertown, MA (US)

(21) Appl. No.: 19/428,965

(22) Filed: Dec. 22, 2025

Related U.S. Application Data
(63) Continuation-in-part of application No. 19/079,849, filed on Mar. 14, 2025.
(60) Provisional application No. 63/902,398, filed on Oct. 20, 2025.

(51) Int. Cl. G05B 19/418; G01N 33/15

(52) U.S. Cl. CPC G05B 19/41845; G01N 33/15

(57) ABSTRACT

The invention provides a modular apparatus comprising at least one process analytical instrument and a controller adapted for fluid communication with a unit operation of a pharmaceutical or biopharmaceutical manufacturing system, systems comprising the modular apparatus, and related compositions and methods. The modular apparatuses described here and associated digital architecture provide a universal solution to fully integrate any pharmaceutical or biopharmaceutical manufacturing process using a wide range of process analytic tools.

### CROSS REFERENCE TO RELATED APPLICATIONS

This application claims priority under 35 USC § 120 as a continuation-in-part of U.S. application Ser. No. 19/079,849 filed Mar. 14, 2025, and priority under 35 USC § 119 as a non-provisional application of U.S. Application No. 63/902,398 filed Oct. 20, 2025, the contents of which are hereby incorporated by reference in their entirety.

### FIELD OF THE INVENTION

The present invention relates to improved methods and arrangements for inline, online, at-line, and offline process analytical technology testing (PAT) boxes, fraction collectors, skids, automated sampling modules, and their associated modules.

### BACKGROUND

Most biopharmaceuticals are manufactured using batch production methods in which human intervention is required to process a set quantity of material to be produced at the same time. Batch operations may require as long as 1-2 months or more from bioreactor to final formulated product. An alternative approach is continuous manufacturing which is attractive due to its potential to reduce costs while increasing productivity and improving product consistency. Continuous manufacturing processes have been developed in the chemical, petrochemical, food, and mechanical industries. In these contexts, continuous processes have demonstrated less reliance on human labor and fewer gaps in transitioning between unit operations in the process resulting in increased productivity, while the smaller facility footprint required by a continuous process reduces facility costs.

There is a need for continuous manufacturing systems in the biopharmaceutical sector, as an alternative to the more time consuming, resource intensive, and expensive batch processes that represent the current standard of practice, as acknowledged by regulatory agencies which have urged the adoption of continuous biomanufacturing in this sector. See National Academies of Sciences, Engineering and Medicine. Continuous manufacturing for the modernization of pharmaceutical production. 2019.

While continuous bioprocessing has yet to be fully realized, several innovations in unit operations have made its implementation more feasible in biopharmaceutical manufacturing. In upstream processing these include developments in perfusion cell culture systems and continuous clarification systems such as continuous centrifugation, alternating tangential flow filtration, and acoustic wave separation. Developments in downstream operations include continuous chromatography and single-pass ultrafiltration and diafiltration capable of achieving high concentration factors and buffer exchange in a single pass of the process material through the module, e.g., in a continuous formulation process.

Despite these and other advances in adapting various unit operations to continuous processing, significant challenges remain in process integration, real-time monitoring and control systems.

### BRIEF SUMMARY

Provided is a customizable, automated, mobile, modular apparatus adapted for performing analytical testing, analysis, and monitoring of a bioprocess in-line or at-line in a biomanufacturing process, and related methods and compositions.

In one aspect, an apparatus for deploying process analytical technology in a pharmaceutical or biopharmaceutical manufacturing process, the apparatus includes process analytical technology hardware in a housing, where the process analytical technology hardware includes a human machine interface, a controller, and a data acquisition and analysis software layer, and an instrument stack to detect data associated with critical quality attributes in samples from the pharmaceutical or biopharmaceutical manufacturing process. The instrument stack includes one or more process analytical instruments coupled with the data acquisition and analysis software layer to provide the data associated with one or more critical quality attributes of the samples. A sample injector to transfer the samples from the unit operation or a recirculation line to the process analytical technology hardware for analysis. The controller includes processing circuitry to execute the data acquisition and analysis software layer to analyze the data collected from the one or more process analytical instruments, to execute one or more instrument models to model the one or more process analytical instruments, and to execute an analysis model to determine information about the one or more critical quality attributes of the samples. The human machine interface communicates the information about the one or more critical quality attributes via a display device. And the process analytical technology hardware includes online process analytical technology hardware, at-line process analytical technology hardware, offline process analytical technology hardware, a fraction collector combined with in-line process analytical technology hardware, or a hybrid thereof. Other technical features may be readily apparent to one skilled in the art from the following figures, descriptions, and claims.

Also provided is a pharmaceutical or biopharmaceutical processing system comprising at least one of the apparatuses as defined above fluidly coupled to at least one of the unit operations of the pharmaceutical or biopharmaceutical manufacturing process. Other technical features may be readily apparent to one skilled in the art from the following figures, descriptions, and claims.

Also provided is a method for real time release testing in a pharmaceutical or biopharmaceutical process, the method includes connecting an apparatus above to a unit operation of the process to capture samples of the process fluid and measuring an indication of at least one product attribute from the process fluid via operation of the one or more process analytical instruments of the process analytical technology hardware. The method further comparing the indication of the at least one product attribute against a predetermined range for the attribute via operation of the data acquisition and analysis software layer and determining whether the indication is within or outside the predetermined range. And the method further executing a first set of instructions via the controller to release the volume of process fluid to a second unit operation in the process if the received indication is within the range, executing a second set of instructions via the controller to release the volume of process fluid to either a recycle flow path or a waste flow path if the indication is outside the range, or executing a third set of instructions via the controller to maintain the volume of process fluid in the recirculation loop until the indication is within the predetermined range. Other technical features may be readily apparent to one skilled in the art from the following figures, descriptions, and claims.

Also provided is a pharmaceutical or biopharmaceutical processing system including a modular apparatus according to any of the preceding embodiments, fluidly coupled between a first unit operation and a second unit operation located immediately downstream from the first unit operation in a biopharmaceutical process.

Also provided is a method for manufacturing a pharmaceutical or biopharmaceutical product that includes integrating a modular apparatus according to any of the preceding embodiments, between one or more unit operations in a batch or continuous processing system. Other technical features may be readily apparent to one skilled in the art from the following figures, descriptions, and claims.

### BRIEF DESCRIPTION OF THE SEVERAL VIEWS OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. In the figures, identical or nearly identical or equivalent elements are typically represented by the same reference characters, and similar elements are typically designated with similar reference numbers, with redundant description omitted. For purposes of clarity and simplicity, not every element is labeled in every figure, nor is every element of each embodiment shown where illustration is not necessary to allow those of ordinary skill in the art to understand the disclosure.

To easily identify the discussion of any particular element or act, the most significant digit or digits in a reference number refer to the figure number in which that element is first introduced.

FIG. 1A is a schematic illustrating the inefficiencies of batch processing in biopharmaceutical manufacturing.

FIG. 1B is a schematic illustrating the advantages of a continuous biomanufacturing process.

FIG. 2 is a schematic illustrating an embodiment of a PAT Box apparatus.

FIG. 3 is a schematic illustrating an embodiment of an in-line flow cell head of a PAT Box apparatus.

FIG. 4 is a schematic showing the flow of data and process material between a PAT Box apparatus and a vessel, recirculation loop, or other process equipment, in accordance with one embodiment.

FIG. 5 is a schematic of a PAT Box apparatus in accordance with one embodiment, showing connection to an external vessel which may be, e.g., a release tank, a hold tank, or a dilution tank such as a flow path connected between the external vessel and the PAT Box apparatus to recirculate process fluid through inline flow cells to measure quality attributes.

FIG. 6 is a schematic illustrating the modular framework of the PAT Box apparatus.

FIG. 7A is a schematic showing data flow between the human machine interface (HMI) and advanced process controller (APC) of the PAT Control Cabinet with the PAT instrument stack containing instruments and associated flow cells, probes, etc. Also depicted is the flow of process fluid in a recirculation loop between the PAT instrument stack and a unit operation such as a reactor or other process equipment, or a vessel, such as a release tank, a hold tank, or a dilution tank.

FIG. 7B is a schematic showing data flow between the HMI and APC of the PAT Control Cabinet with two PAT instrument stacks each connected to a different unit operation or vessel.

FIG. 8 is a schematic illustration in accordance with one embodiment showing flow cells of the PAT Box apparatus in a series and parallel configurations.

FIG. 9 is a schematic illustration in accordance with one embodiment showing examples of flow cells for in-line process analytic instruments and sample ports and cuvettes for at-line instruments.

FIG. 10A is a schematic in accordance with one embodiment showing a PAT Box apparatus connected in-line between two unit operations for monitoring a continuous process. As pictured, the flow cells are integrated in parallel in the connection path.

FIG. 10B is a schematic illustration of a PAT Box apparatus in accordance with one embodiment, showing the PAT Box connected to a single unit operation in a recirculation flow path where the flow cells are integrated in parallel.

FIG. 11 is a schematic illustration of a PAT Box apparatus showing the PAT Box connected in-line between two unit operations for monitoring a continuous process, as in FIG. 10A, with the addition of a waste valve and a forward processing valve.

FIG. 12A is a schematic illustration of a PAT Box apparatus showing a configuration for real time release testing between two unit operations in a continuous process with a waste collection vessel, waste valve, forward processing valve and reagent return/material recycling valve.

FIG. 12B is a schematic illustration of a PAT Box apparatus showing another configuration for real time release testing where the apparatus is positioned at a first unit operation in the continuous process with a waste collection vessel, waste valve, forward processing valve and reagent return/material recycling valve.

FIG. 13 is a schematic illustrating an exemplary biopharmaceutical manufacturing system in accordance with one embodiment, showing several PAT Box apparatuses connected in-line between unit operations for monitoring a continuous process.

FIG. 14 is a schematic illustrating an exemplary biopharmaceutical manufacturing system in accordance with one embodiment where several PAT Box apparatuses are connected between unit operations via a vessel, such as a surge vessel.

FIG. 15 is a schematic illustrating an exemplary biopharmaceutical manufacturing system in accordance with one embodiment where several PAT Box apparatuses are connected between unit operations via a vessel, such as a surge vessel and another PAT Box apparatus is situated in-line between unit operations.

FIG. 16 is a schematic diagram illustrating the possible use by the PAT Box apparatus of in-line, at-line, and off-line process analytical data.

FIG. 17 is a graph illustrating raw spectra collected from an in-line Raman flow cell.

FIG. 18 is a graph illustrating partial least squares regression (PLS) model predictions for mRNA concentrations versus off-line ultraviolet-visible (UV-VIS) spectrophotometer measurements for the in vitro transcription (IVT) reactor test operation.

FIG. 19 is a graph illustrating PLS model predictions for the IVT test operation.

FIG. 20 illustrates a plot of in-line FlowVPX measurements vs off-line UV-VIS measurements at a first tangential flow filtration (TFF) operation, TFF1 pool.

FIG. 21 illustrates a plot of in-line FlowVPX measurements vs off-line UV-VIS measurements at OdT pool.

FIG. 22 illustrates a plot of in-line FlowVPX measurements vs off-line UV-VIS measurements at a second TFF operation, TFF2 pool.

FIG. 23 illustrates raw spectra collected from in-line Mid-IR flow cell in the IVT run.

FIG. 24 illustrates a PLS model training plot showing alignment of off-line UV-VIS measurements to the PLS model predictions.

FIG. 25 illustrates a PLS model prediction of all measurements taken during CP1-Fluc-005 vs off-line UV-VIS measurements.

FIG. 26 illustrates results from Waters' MLAS system being used to measure particle average size, concentration, and Z-average radius for lipid nanoparticle formulation (LNP) samples.

FIG. 27 illustrates a control graph of LS11 (talaporfin sodium).

FIG. 28 illustrates results of a NanoFlowSizer being used to measure Z-average and polydispersity index (PDI) for the T2 LNP sample.

FIG. 29 illustrates results of a NanoFlowSizer being used to measure Z-average and PDI for the T3 LNP sample. The results are in good alignment with the off-line dynamic light scattering (DLS) measurement using Malvern systems.

FIG. 30 illustrates a PAT Box Apparatus monitoring a bioprocess product flow stream.

FIG. 31 illustrates a PAT Box Apparatus monitoring quality attributes and reaction kinetics of bioprocess reaction vessel containing reagents and buffer that produce active product ingredients and/or drug substances.

FIG. 32 illustrates PAT Box Apparatus monitoring quality attributes of a bioprocess product intermediate bulk in a surge vessel or other intermediate holding container typically positioned between unit operations.

FIG. 33 illustrates differences between in-line, online, at-line, and offline analytics in accordance with one embodiment.

FIG. 34 illustrates various PAT boxes in accordance with one embodiment.

FIG. 35 illustrates various hybrid PAT boxes in accordance with one embodiment.

FIG. 36 illustrates various offline PAT boxes in accordance with one embodiment.

FIG. 37 illustrates an automated pharmaceutical or biopharmaceutical process system comprising various PAT boxes of in accordance with one embodiment.

FIG. 38 illustrates an online PAT box in accordance with one embodiment.

FIG. 39 illustrates an at-line PAT box in accordance with one embodiment.

FIG. 40 illustrates a workflow with offline PAT boxes in accordance with one embodiment.

FIG. 41 illustrates an in-line PAT box with fraction collection in accordance with one embodiment.

FIG. 42 illustrates an in-line/online hybrid PAT box in accordance with one embodiment.

FIG. 43 illustrates an in-line/at-line hybrid PAT box in accordance with one embodiment.

FIG. 44 illustrates an in-line/online/at-line hybrid PAT box in accordance with one embodiment.

FIG. 45 illustrates an in-line/at-line/offline hybrid PAT box in accordance with one embodiment.

FIG. 46 illustrates a workflow of an in-line PAT box with an automated sampling deck and offline PAT boxes in accordance with one embodiment.

FIG. 47 illustrates a workflow for an in-line PAT box skid with automated sampling deck connected to modular offline sample injection stack skids and offline PAT box skids in accordance with one embodiment.

FIG. 48 illustrates an embodiment of a process as a flowchart in accordance with one embodiment.

FIG. 49 illustrates an embodiment of the PAT box skid that allows for multiple reaction vessels to be sampled and directed to an inline, at-line, on-line, offline, or fraction collector analytical device for measurement of quality attributes.

FIG. 50 shows multiple figures that outline a Raman spectrophotometer model for measuring glucose in monoclonal antibody (mAb) manufacturing process.

### DETAILED DESCRIPTION

Embodiments advantageously address the need for improved process analytic technology (PAT) and universal integration to address the needs of a continuous pharmaceutical and biopharmaceutical manufacturing system. The disclosure provides a customizable, automated, mobile, modular apparatus for deploying process analytical technology (PAT) in a continuous biopharmaceutical manufacturing process, referred to herein as a PAT Box apparatus. The apparatus is customizable and modular in terms of its ability to be adapted to monitor different unit operations and be deployed in a “plug and play” fashion at single or multiple locations in a continuous biopharmaceutical manufacturing process. These features allow for customizable real-time data acquisition and monitoring of one or multiple unit operations in the process.

The PAT Box apparatus described herein advantageously combines multiple process analytical technologies into a single contiguous flow path for in-line and/or at-line monitoring of a biomanufacturing process at one or multiple points in the process. The customizable, mobile and compact structure of the PAT Box apparatus obviates the need to configure different analytical technologies in a separate location, such as a cleanroom or distant lab bench. Instead, the PAT Box apparatus consolidates the technologies into, e.g., a mobile cart or other housing where their respective flow cells are arranged into one contiguous flow path for analysis and monitoring. This arrangement significantly increases flexibility in part by providing flow kits adapted for different technologies that can easily be swapped in and out depending on process needs. Process efficiency is enhanced by bringing the analytics to the unit operations in-line, online, offline, at-line, or hybrids thereof. Further efficiency improvements are achieved by the decreased sample volumes required for the analytical measurements performed by the consolidated technologies and single flow path of the PAT Box apparatus, particularly where the sensors and/or flow cells are situated in-line in a recirculation loop with the process. Hold-up volumes are also substantially reduced by the ability to bring the apparatus into close proximity with the unit operations. In addition, sample handling is reduced or even eliminated because the mobile PAT Box apparatuses can be moved as needed to different unit operations in the process. The reduction in sample handling also reduces contamination risks. Proximity to the unit operations and/or the use of in-line recirculation loops further increases efficiency by substantially reducing the time otherwise needed to remove a sample from the process, transfer it to an analytic instrument or multiple different analytic instruments, and perform associated tasks of sample management, such as manual data entry of sample identifiers (IDs), forms, etc. Overall, the PAT Box apparatus as described herein provides for the ability to automatically obtain in-process analytics and release test results in real-time to increase process efficiency by reducing both costs and production time. In some cases, where product may be lost due to instability during prolonged processing times using standard methods product quality and yield may also be improved in accordance by incorporation of a PAT Box apparatus into the process as described here.

In one aspect, the PAT Box apparatus comprises PAT hardware such as a PAT instrument stack, a PAT sample injector, a PAT sample receiver, a fraction collector, a sample collector deck, and a PAT Control Cabinet. The PAT instrument stack includes in-line, online, at-line, and offline instruments and accompanying flow cells, valves, tubing, and pumps, as well as optional probes, cuvettes, and sampling ports such as sample receivers and sample injectors that may be utilized, for example, for offline process analytical technologies. The apparatus also includes electronic connectors adapted to connect the instruments directly to the control cabinet and fluid connectors to connect the flow cells to the process flow path.

The PAT Control Cabinet may comprise a human machine interface (HMI), a controller, such as an advanced process controller (APC), and at least four software layers including data acquisition, process scheduling, deviation handling, and real-time execution layers. The data acquisition layer acquires and stores data from the in-line, online, at-line, and offline instruments, or a combination thereof in hybrid PAT boxes. In aspects, the data acquisition layer also functions to close a control loop by communicating with a distributed control system (DCS) or Supervisory Control and Data Acquisition (SCADA) system. In aspects, the data acquisition layer may also report process data to historians and other databases. In aspects, the data acquisition layer may include additional software for performing process modeling such as chemometric modeling and/or other advanced process modeling, for example to process the raw data into product attribute information, which may include for example critical quality attribute (CQA) information or product quality attribute information.

In use, each PAT Box apparatus brings PAT analytics to one or more unit operations in a pharmaceutical or biopharmaceutical manufacturing process enabling product attributes to be monitored, controlled and “released or rejected” for a volume of process fluid at any of several critical control points (CCPs) in the process. The terms product attribute, product quality attribute (PQA), and critical quality attribute (CQA) are used interchangeably herein to refer to a physical, chemical, or biological property or characteristic that should remain within a predetermined limit, range, or distribution to ensure a desired product quality.

In aspects, a PAT Box apparatus is customized for monitoring and control of a particular unit operation in a manufacturing process. A PAT box skid may include one or more PAT boxes as well as one or more vessels for storage of samples. In many embodiments, the one or more vessels may reside in the housing of the PAT box or PAT box skid A unit operation may include, for example, a bioreactor process, a chromatographic separation process, or a filtration process such as a tangential flow filtration (TFF) process or an ultrafiltration process. The particular unit operation will depend upon the process. For example, unit operations in a monoclonal antibody production process may include a bioreactor, a clarification unit which may be, e.g., a filtration unit such as a depth filtration unit, affinity chromatography, viral inactivation, polishing chromatography using e.g., ion exchange (Cation-exchange chromatography, CEX or Anion-exchange chromatography, AEX) or hydrophobic interaction chromatography (HIC), additional filtration units including diafiltration and sterile filtration, as well as fill and finish units. Similarly for other processes such as plasmid deoxyribonucleic acid (DNA) production, adeno-associated virus (AAV) production, and ribonucleic acid (RNA) production, the unit operations may include a cell culture bioreactor, a fermentation reactor, or an in vitro transcription (IVT) reactor along with one or more chromatography units and filtration units as well as specialized units such as a lipid nanoparticle formulation (LNP) unit. Accordingly, a PAT Box apparatus as described here may be used in various biopharmaceutical manufacturing processes including processes for manufacture of monoclonal antibodies, antibody drug conjugates, vaccines, including RNA vaccines, RNAi, enzymes, peptides, cell therapies, viral vectors including adeno-associated virus (AAV) vectors and lentivirus vectors, and other gene therapy modalities. In aspects, the manufacturing process is a continuous process. In aspects, the manufacturing process is a continuous process for manufacture of RNA, adeno-associated virus, plasmid DNA, or monoclonal antibodies. In further embodiments, the PAT box or PAT box skid may include with an instrument stack and an HMI with a data acquisition and analysis software layer configured for water purification and buffer manufacturing.

Additional flexibility of the PAT Box apparatus is provided in part by its digital infrastructure which allows for the processing of raw data from various process analytic instruments, which may be in-line, at-line, online, or off-line, into process-specific product quality attributes. In aspects, the digital infrastructure includes at least one model based on underlying data and assumptions for a particular unit operation. The model may be a mechanistic model or a data-driven model, or a hybrid model that combines aspects of both mechanistic and data-driven models. A mechanistic model refers to a model based on biophysical relationships that have been mathematically elucidated based on a full mathematical understanding of the process and may also be referred to as “white-box models”. Data-driven models are mathematical models based solely on the statistical relationships between data, primarily data obtained or derived from online sensors and offline analysis. Data-driven models are not based on biophysical relationships and may also be referred to as “black-box models.” Hybrid models combine mechanistic and data-driven models. The digital infrastructure may also include algorithms to measure the relationship between variables using correlation analysis which relies on establishing correlations between sensor signals, process parameters, and quantity and quality parameters which may be measured offline. For example, the extent of the linear relationship is determined using a Pearson's correlation. Other methods are available to measure nonlinear relationships, for example, Spearman's rank correlation, which is a nonparametric measure of rank correlation reporting the statistical relationship between the rankings of two variables. In aspects, the digital infrastructure may include algorithms for carrying out one or more statistical methods selected from multiple linear regression (MLR), partial least squares regression (PLS), structured additive regression (STAR), random forest (RF), support vector machines regression (SVM), neural networks (NNs), deep learning (DL), Gaussian process regression (GPR), as well as other machine learning models. In some embodiments, models may include digital twins and process simulators for simulating portions of and/or the entire pharmaceutical or biopharmaceutical manufacturing process.

Accordingly, also provided are methods for the design, optimization, monitoring, and control of a biopharmaceutical manufacturing process utilizing a PAT Box apparatus as described herein, as well as related systems incorporating at least one PAT Box apparatus.

In an aspect, provided are methods for designing and/or optimizing a biopharmaceutical manufacturing process utilizing one or more PAT Box apparatuses as described herein to identify critical control points (CCPs) in the process, i.e., the critical unit operations at which product quality attributes should be monitored for real-time testing and release at each unit operation.

In an aspect, provided are methods for monitoring and controlling a biopharmaceutical manufacturing process utilizing one or more PAT Box apparatuses as described herein, the methods comprising continuous monitoring and control of one or more critical quality attributes (CQAs) in a process stream by operation of a PAT Box apparatus as described herein.

In an aspect, provided are methods for real-time monitoring, testing, and release of a product stream at one or more defined points in a biopharmaceutical manufacturing process, which method may also be referred to as real time release testing (RTRT), the methods comprising monitoring, testing and release by operation of a PAT Box apparatus as described herein.

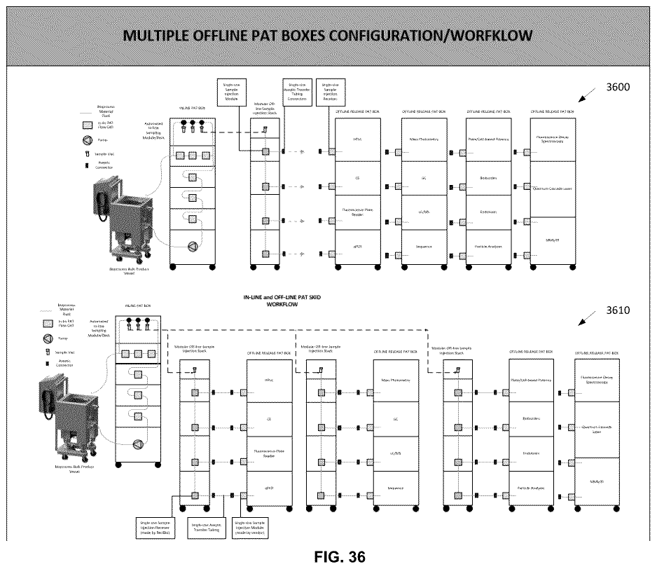

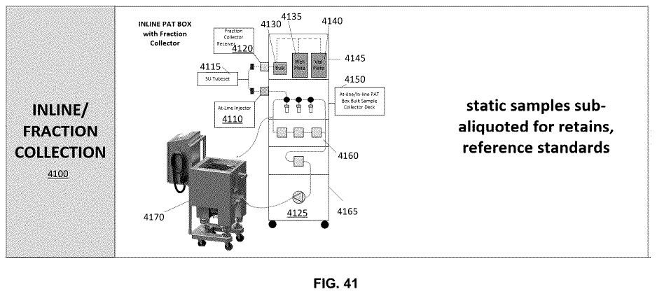

A purpose of the online, at-line, offline and hybrid PAT Boxes and PAT box skids and their associated modules is to facilitate transfer of bioprocess material samples from the manufacturing line to virtually any analytical system in near real time. This solves multiple problems currently facing the biopharmaceutical industry with analytical and quality control testing that requires samples to be manually taken from the process, handled, transferred, and submitted to analytical systems typically found in separate rooms or buildings than the drug manufacturing process room or facility. Embodiments of the online, at-line, offline, and hybrid PAT Boxes and PAT box skids essentially make it possible to bring virtually any analytical system needed to measure attributes of a manufacturing process and physically connect it to the manufacturing process through hardware and software automation. Example technical problems advantageously solved by embodiments include but are not limited to:
Reduced quality control lab space for at-line and off-line instrument installations space, thus reducing facility operating expense costs.
Reduced operating cost of quality control technician labor to run analytical instruments
Reduced sample handling errors by removing the human component and automating the sample transfer in a closed environment, thus reducing failed analytical tests and reducing sample volume need to retain, store samples, cost of time to retest samples, and cost of manufacturing down time.
Reduced manufacturing times while Manufacturing teams wait on quality control (QC) to transport samples to the QC lab, test the samples, and report results to Manufacturing. A Process Control System (PCS) and PAT Software under single platform, including the online, at-line, offline, and hybrid PAT boxes and skids and modules, makes QC result reporting and forward progressing manufacturing seamless; while reducing hold time. Reduced manufacturing time, thus, increases the number of batches produced over time in a facility, increases facility productivity, increases speed to market and patient, and increases business profits.
Compared to in-process control (IPC) testing (analytical measurements completed in between unit operations before a release point) which can largely be measured by in-line PAT, release testing of quality attributes still requires online, at-line, and offline analytical systems to measure a significant percentage of the release quality attributes. Without an online, at-line, and/or offline PAT Box, this creates a gap for real time release of manufacturing material at the, for example, active pharmaceutical ingredient (API), drug substance (DS), and drug product (DP) release points. To make real time release a reality, there must be an interface between the manufacturing bioprocess release material and the online, at-line, and offline analytical instruments. The online, at-line, offline, and hybrid PAT boxes and skids solve this problem.
The online, at-line, offline, and hybrid PAT boxes and skids may also come equipped with a fraction collector deck. Like the problem with collecting release samples and manual transport to quality control (QC), collecting retain samples for stability studies, reference standards, etc. is manual and error prone. Fraction collectors, bulk sample collectors, and liquid handlers may also be needed for sample preparation for at-line and offline release testing. The fraction collector deck may be mounted adjacent to the bioprocess fluid path and may pull samples as needed while bioprocess fluid runs through the other decks of the PAT Box or PAT box skids, see FIG. 36 and FIG. 41.

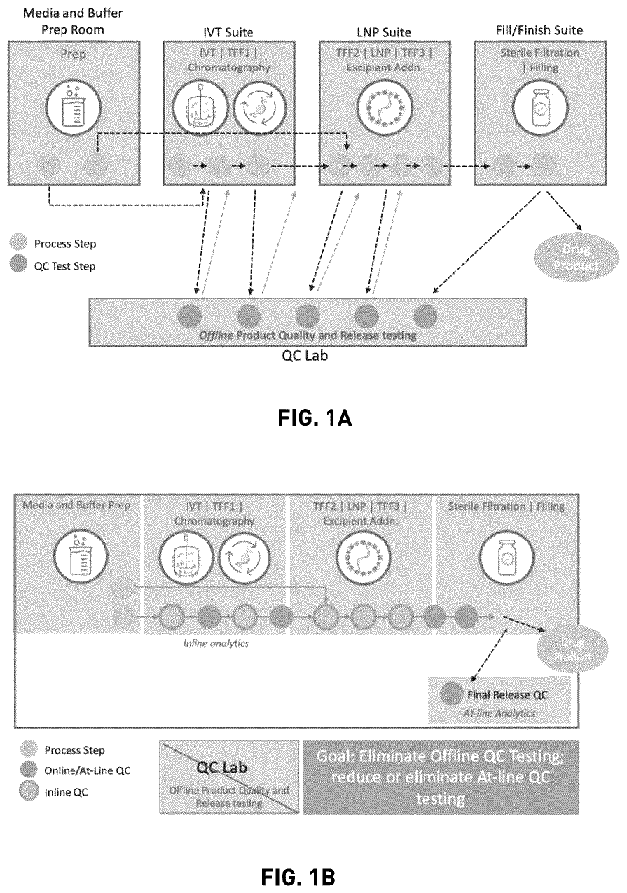

The advantages of the PAT Box apparatus described here include optimizing a biopharmaceutical manufacturing process for continuous operation via monitoring and control of one or more unit operations in the process. FIG. 1A illustrates the disadvantages of batch processing where at least several analytical assays must be conducted in an off-line setting. This requires samples to be removed from the batch process and stored until data is collected and a determination is made as to whether quality control (QC) standards are met or not. Process steps are gated by QC release. This creates inefficiencies due to the need to transfer and test samples taken at various points in the process and hold until QC release. This is illustrated in the figure by dashed lines which represent the broken progression of a biopharmaceutical product along the production line due to the need to wait for off-line QC testing results in order to move forward. In addition, limited in-process information is obtained, increasing risk that the manufacturing process may need to be reworked.

FIG. 1B illustrates a continuous biomanufacturing process in which QC testing occurs during the process, for example using a PAT Box apparatus as described herein. Solid lines in the figure illustrate the uninterrupted progression of product along the production line. Continuous manufacturing has several advantages including uninterrupted manufacturing whereby QC results are fed to automated control software allowing for constant monitoring and real-time product characterization, and all processes and QC testing occur in a single cleanroom suite. Generally, utilizing a continuous process, product can be produced over 24-48 hours compared to on the order of 2 or 3 months for a batch process.

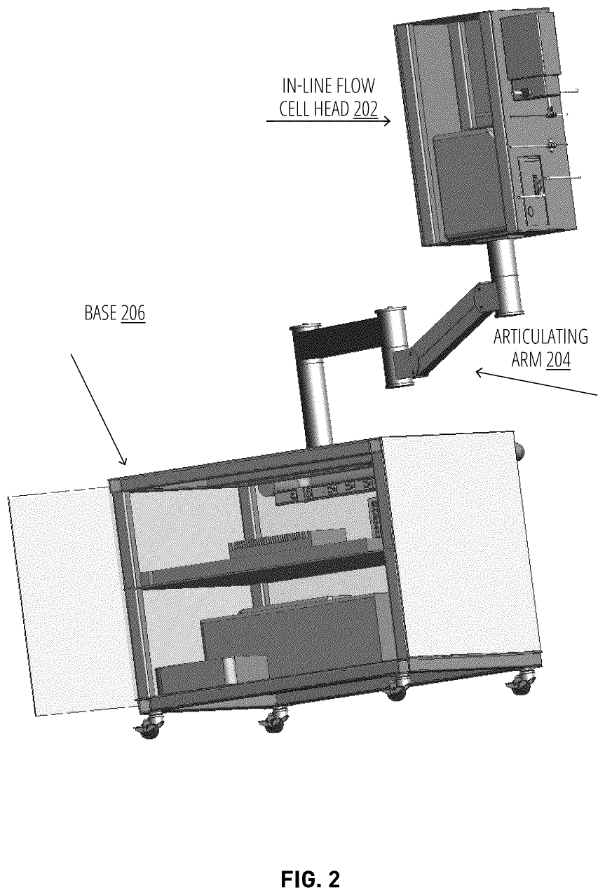

FIG. 2 illustrates an embodiment of a PAT Box apparatus where a weighted base 206 houses the PAT Control Cabinet and the PAT instrument stack. An articulating arm 204 connects the weighted base to an in-line flow cell head 202 containing a set of flow cells customized for process-specific in-line instruments and their respective transmitters. The PAT instrument stack contains the in-line instrument computers. The PAT Control Cabinet contains electrical I/O, and optionally a programmable logic computer (PLC). The control cabinet may also contain an ethernet switch for connecting to a manufacturing network and/or a digital control infrastructure that may include e.g., a digital twin, a simulator, process models, and a Knowledge Hub which may integrate facility data with process data, including historical process data and clinical data, as well as data from the digital twin and/or simulator. The PAT Box apparatus may also include tubing, valves and pumps configured to control the flow of process fluid between an external vessel, a unit operation, and/or a recirculation loop and the flow cells. In operation, one or more valves and/or pumps may be controlled by a controller of the PAT Control Cabinet in accordance with one or more predetermined criteria. The PAT Box apparatus may also include fluid inlet and outlet ports, additional ports for sensors and/or probes and one or more sample ports for data acquisition and material analysis. Also provided is a system including a PAT Box apparatus and associated mechanical and digital infrastructure.

The in-line flow cell head 202 is adapted to add or remove flow cells as needed to customize the PAT Box apparatus for use with a particular unit operation. The flow cell head may also include one or sample ports and/or cuvettes. This versatility allows for the incorporation of data from a variety of different in-line, at-line, and off-line process analytical instruments. Exemplary process analytical instruments may include spectrophotometers, refractometers, dynamic light scattering instruments, nuclear magnetic resonance (NMR) instruments, liquid chromatography-mass spectroscopy (LC-MS) instruments, and the like. Thus, in one aspect, the PAT Box apparatus may be configured to measure and process data from one or more spectroscopic instruments, such as instruments for detecting Raman, infrared (IR), and ultraviolet (UV) spectra. In another aspect, the PAT Box apparatus may be configured to measure and process data from both a spectroscopic instrument and one or more additional instruments such as a refractometer for measuring refractive index (RI), and/or a dynamic light scattering (DLS) instrument, e.g., for measuring multiangle light scattering (MALS), which may also be referred to as a MALS detector. In an aspect, the PAT Box apparatus is configured to support an at-line process analytical instrument, optionally wherein the at-line process analytical instrument is a high performance liquid chromatography (HPLC) instrument, a flow cytometer, a capillary electrophoresis (CE) instrument, a mass spectrophotometer, an osmometer, a UV-VIS spectrophotometer, a fluorometer, a light scattering detector, or a luminometer.

The articulating arm advantageously brings the in-line flow cells close to the unit operation or vessel in the biomanufacturing process. In some aspects, a recirculation line may be used to pull process material from the unit operation for passing through the in-line flow heads, sensors, and/or probes of the head 202. Use of an articulating arm allows for a shorter recirculation line and smaller hold-up volumes in the line compared to what would be required if the apparatus was located at a more distant site from the unit operation. The articulating arm also allows for optimal positioning and increased mobility around the manufacturing equipment while decreasing the overall footprint needed for the PAT Box apparatus.

Some configurations of the PAT Box apparatus may require fewer process analytic technologies or technologies that have smaller components. Combined with an articulating arm, this may create an unstable top-heavy configuration unless properly mitigated with a weighted base. Thus, the weighted base provides an additional functional enhancement to increase safety to equipment and personnel.

In addition, some configurations of the PAT Box apparatus may include built-in environmental protections such as electromagnetic frequency (EMF) shielding, vibrational dampening, shock absorption, and temperature control. For example, where the PAT Box apparatus includes a Mid IR and NMR component, EMF shielding can be utilized to protect the instrument from interference that could cause failure or inaccurate measurement. As another example, where a PAT Box apparatus includes a DLS component, vibrational dampeners and shock absorbers may be included. Temperature control strategies may include one or more air fans or liquid cooling using convective heat transfer.

In aspects, the weight base comprises an automated leveling system.

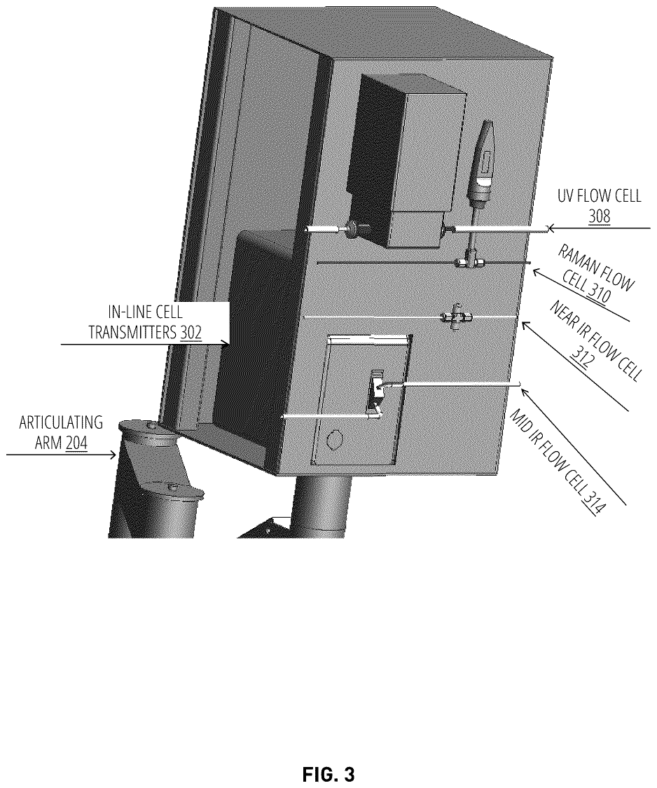

FIG. 3 is an inset of the in-line flow cell head 202 showing exemplary flow cells, a UV flow cell, a Raman flow cell, a Near IR flow cell, and a Mid IR flow cell, secured to the flow cell head. The interior of the head contains any necessary transmitters for the in-line flow cells. This configuration allows for the flow cells to be positioned close to a unit operation or vessel recirculation loop for fluid connection in-line to the biomanufacturing process.

In aspects, the PAT Box apparatus may be configured to measure and process data from one or more of a spectrophotometer, a refractometer, a multiangle light scattering (MALS) detector, and/or a DLS instrument. In aspects, the spectrophotometer may detect Raman spectra, including Fourier Transform Raman Spectroscopy (FR-Raman), infrared (IR), including mid-IR (MIR), near-IR (NIR), far-IR (FarIR), or ultraviolet (UV) spectra and/or visible (VIS) wavelengths. In aspects, the spectrophotometer may also include an ion mobility spectrophotometer (IMS).

In aspects, the instrument stack of the PAT Box apparatus includes two or more spectroscopic instruments selected from a Raman spectrophotometer, a mid-infrared (IR) spectrophotometer, a near-IR spectrophotometer, and an ultraviolet-visible (UV-VIS) spectrophotometer.

In aspects, the instrument stack of the PAT Box apparatus includes a Raman spectrophotometer, a mid-infrared (IR) spectrophotometer, a near-IR spectrophotometer, and a UV-VIS spectrophotometer. In aspects, the PAT Box apparatus includes a Raman spectrophotometer, a mid-infrared (IR) spectrophotometer, a near-IR spectrophotometer, a UV-VIS spectrophotometer and a multiangle light scattering (MALS) detector.

In aspects, the instrument stack of the PAT Box apparatus includes a refractometer.

In aspects, the instrument stack of the PAT Box apparatus includes an ion mobility spectrophotometer (IMS).

In aspects, the instrument stack of the PAT Box apparatus includes a nuclear magnetic resonance (NMR) instrument.

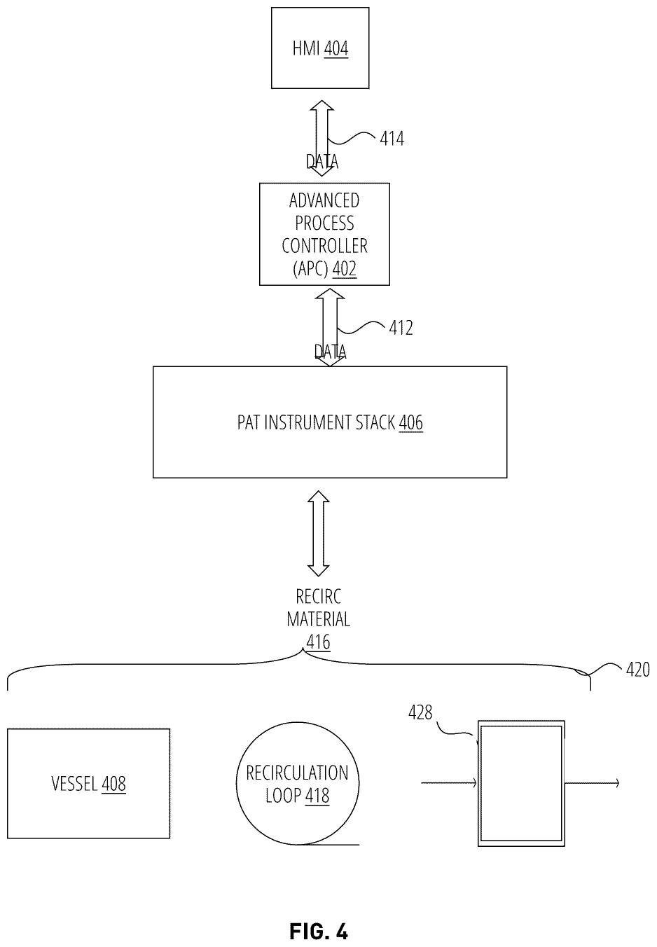

FIG. 4 schematically illustrates the flow of data and process fluid in accordance with different aspects of a PAT Box apparatus in operation. Shown are the HMI 404 and controller, e.g., an Advanced Process Controller (APC) 402 of the PAT Control Cabinet. Also illustrated is the flow of data between these two elements and the PAT Instrument Stack 406. Also shown is the flow of process fluid as Recirc Material 416 between the flow cells, sensors, and/or probes of the PAT Instrument Stack 406 and an external vessel 408, a recirculation loop 418, or a unit operation 428.

In an illustrative example of real time release testing (RTRT), the vessel 408 may be fluidly connected with a process stream between a first unit operation and a second unit operation in a biopharmaceutical manufacturing process. In operation, a volume of the process stream enters the vessel 408 from the first unit operation and recirculates between the vessel and the flow cells, sensors and/or probes of the PAT Box apparatus while data is obtained. Data obtained from the PAT instruments is analyzed by the (APC) 402 operating in conjunction with an appropriate model to determine if the product stream satisfies a predetermined criteria, which may relate to, for example, a CQA of the product. If the criteria is satisfied, the controller executes a set of instructions to release the volume of process fluid to the second unit operation in the process.

The HMI may include a digital display comprising a dashboard including a visual output of model results and an interface for control of individual sensors and probes. In aspects, the HMI is in the form of a computer, for example a laptop computer or a programmable logic computer (PLC).

As discussed above, in aspects, the controller may include at least four software layers. The at least four software layers include a data acquisition layer, a process scheduling layer, a deviation handling layer, and a real-time execution layer. In aspects, the software layers execute a set of programmable instructions. In aspects, the programmable instructions may be programmed in a language selected from C, Python Java, JavaScript, Perl, Tcl, or Smalltalk.

In aspects, the controller includes a distributed control system (DCS) including a host computer performing optimization algorithms and advanced control strategies and one or more proportional integral derivative (PID) controllers performing device level controls. The system may also include control units performing regulatory level control functions, such as PID algorithms, and may also include data gathering and extraction capabilities. The system may also include data storage devices to store process data for control and process analytics. Also included is software to communicate and interact with controllers, inputs, and outputs.

In aspects, the APC and HMI may be situated in a cabinet physically separated from the PAT Instrument Stack 406.

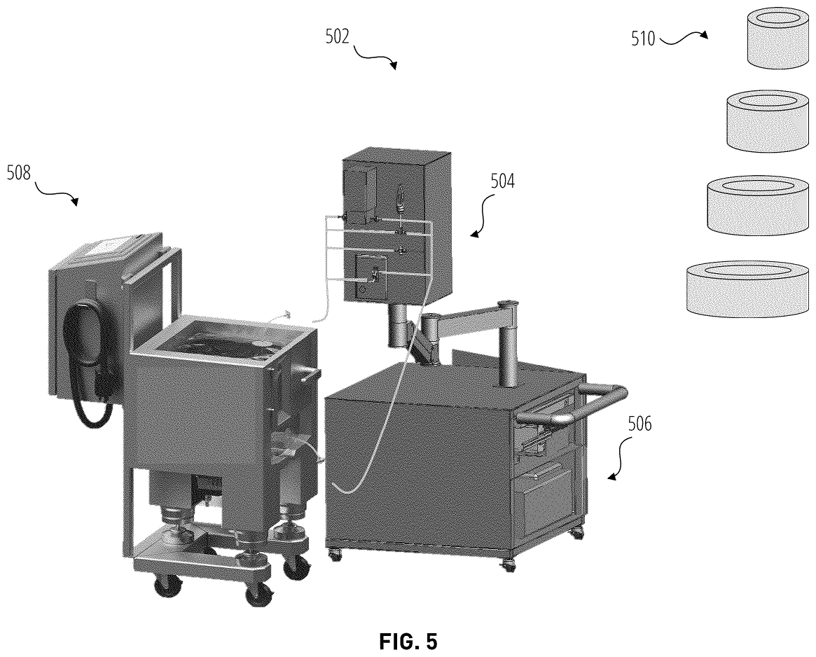

FIG. 5 illustrates a configuration of a PAT Box apparatus 502 situated next to an external vessel 508 which may be connected to a unit operation of a manufacturing process, or may be situated in-line between two unit operations of the process. The figure illustrates tubing connecting the flow cells of the head 504 to the external vessel. In other aspects, where the external vessel is absent, the tubing may connect directly to a unit operation of the process. The external vessel may be any type of vessel, tank or bag. The external vessel may include a plurality of ports, including IO ports for data transfer between multiple control panels and/or software. In aspects, the external vessel incorporates one or more analytical detectors, sensors and/or probes including for example, a conductivity sensor, a temperature sensor, and/or pH probe. The external vessel may have a volume of from 0.100 to 5000 liters, from 0.500 to 1000 liters, or from 2 to 500 liters. The list of range of volumes is not intended to be limiting and can fall below 0.1 liters and above 5000 liters.

### Flow Kits

In an advantageous aspect of its versatility and modularity, a PAT Box apparatus may be configured with a custom set of flow cells and optional sensors or probes, along with associated tubing and valves adapted for a particular set of process analytic instruments to meet the needs of a particular manufacturing process. The set of flow cells, tubing, valves and optional sensors or probes adapted for a specific use may be referred to herein collectively as a flow kit.

The tubing accompanying a flow kit may be adapted for various purposes such as temperature control and/or flow rate. In aspects, the temperature control capabilities may derive from insulated tubing and/or a jacketed stainless steel tube with temperature control fluid. In the latter context, the outer tube flows temperature controlled fluid that is regulated by a temperature control unit (TCU). The inner tube contains the product flow stream and temperature control fluid passes around the product flow stream thereby transporting heat towards or heat away from the product flow stream via convection. The temperature control fluid that transports heat towards or away from the product flow stream then returns to the TCU to be refreshed towards the temperature set-point. This aspect may be useful, for example, where product is temperature sensitive and/or temperature variation can cause fouling of the tubing or sensors.

The tubing accompanying a flow kit may also or alternatively be adapted to produce a particular flow rate through the flow cell(s). In FIG. 5, the inset 510 illustrates the wide range of flow rate capabilities that may be obtained using tubing having various different inner diameters. For example, specific flow rates, pressure profiles, or Reynolds number conditions can be attained inside the tubing or flow cell by either increasing or decreasing the flow rate via the in-line flow cell tubeset pump, and/or by increasing or decreasing the tubing inner diameter. In aspects, the flow rate may range from 0.0-50.0 L/min and the inner diameter of the tubing may range from 1/32 inch to ½ inch. In aspects, the inner diameter of the tubing may be 1/32 inch, ⅛ inch, ¼ inch, or ½ inch, but is not limited to these sizes.

Also provided are additional flow kits including a calibration flow kit. The calibration flow kit is used to calibrate in-line flow cell instrumentation and/or validate the operational and performance of the in-line flow cell instruments. For example, a Raman in-line flow cell may need to be tested to confirm the laser intensity is within range and the spectral readings are sending back the range and intensity as expected for a certain reference standard such as ethanol. This calibration activity might be performed before first use, such as during an installation, operation, and/or performance qualification or for routine preventative maintenance activities. After completion of calibration or validation activities, the calibration flow kit may be removed and replaced with a process flow kit.

Also provided is a cleaning in place (CIP) flow kit. Where in-line flow cells are permanently attached to their internal components or instrumentation and cannot be made single use, the flow cells may need to be cleaned and sanitized, either before use or after becoming fouled. The CIP flow kit includes “Y” connectors upstream and downstream of the flow cells so that cleaning solution can flow through the flow cells.

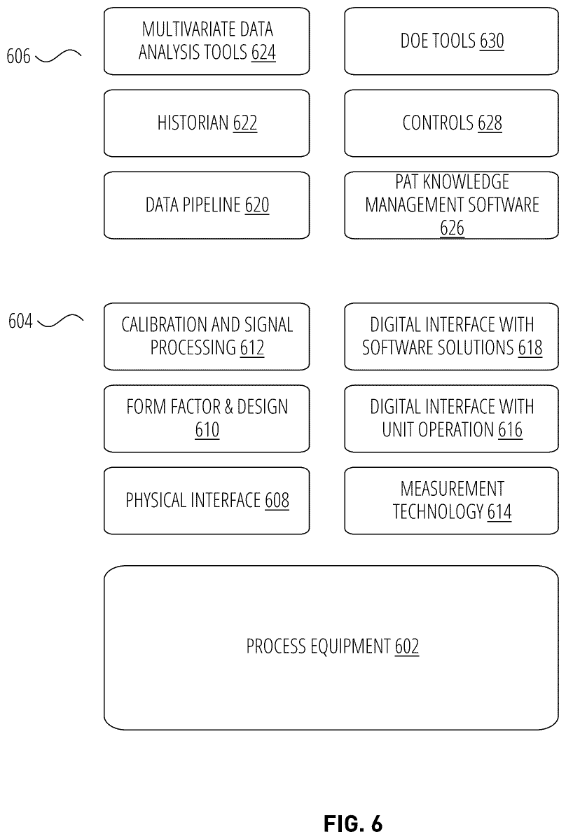

FIG. 6 schematically illustrates an embodiment of a PAT Box apparatus as described herein. The figure illustrates the modular framework of the apparatus. Shown is the PAT instrument stack 604 and the PAT Control Cabinet 606 of the apparatus which integrate with process equipment 602.

The PAT Control Cabinet includes communications, data, and analysis (CDA) software. In aspects, the PAT Control Cabinet includes computational modeling 624 software such as multivariate data analysis tools 624 which permit real-time insight and response via accessible/static and dynamic model building. In aspects, also included is design of experiments (DOE) software, a historian 622, process controls 628, and a data pipeline 620. Also included is PAT Knowledge Management software 626 which provides automated data collection, extraction, harmonization and storage as well as multivariate data analysis (MVDA) capabilities, technology transfer and batch record integration. In aspects, an application programming interface (API) adapter may be included to integrate with existing in-line or at-line process analytic instruments. In operation, the PAT Knowledge Management software 626 processes raw data, including using available models as needed to convert the data, e.g., spectra, into appropriate product attributes, including critical quality attributes (CQAs). In aspects, software tools of the PAT Control Cabinet provide easy to understand outputs having clear direction that may be integrated into process controls.

The PAT instrument stack 604 includes various modules adapted to be quickly integrated into a manufacturing process in a “plug and play” fashion. In aspects, the modules include a physical interface 608 with the process equipment. In aspects, the interface is fully integrated with a single-use system comprising single use pre-calibrated consumables in a closed, sterile system. Another module includes scalable in-line and on-line process measurement technologies 614 which measure CCPs and CQAs for process control and release testing. Also included is a calibration and signal processing 612 module. In aspects, the calibration and signal processing 612 module is pre- or auto-calibrated. In aspects, the calibration and signal processing 612 module includes straightforward calibration workflows not disruptive to the process and/or where limited recalibration is required.

In aspects, the PAT Box apparatus may track in real-time the progress of a process and transmit data until a desired optimization is reached or the process terminates.

In aspects, the PAT Box apparatus is configured to control upstream and downstream unit operations via included compatibility with industry standard systems including, for example, Delta V™, Ignition™, Unicorn™, Wonderware™ MFCS™, and OPC-UA™. In aspects, the PAT Box apparatus controls upstream and downstream unit operations based on analytical results gathered by all process analytic instruments, e.g., in-line, at-line, on-line, and off-line.

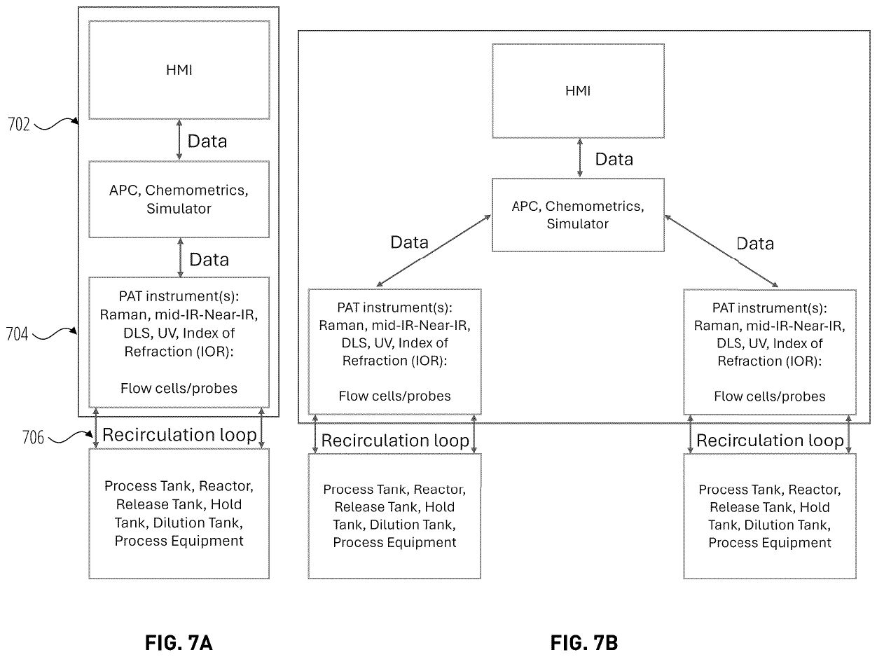

FIG. 7A schematically illustrates PAT Control Cabinet 702 including a human machine interface (HMI), a controller, depicted as an advanced process controller (APC), and software layers including a data acquisition layer that acquires and stores data from the in-line and at-line instruments, as well as any optional off-line instruments. In aspects, the data acquisition layer also functions to close a control loop by communicating with a distributed control system (DCS) or SCADA system. In aspects, the data acquisition layer may also report process data to historians and other databases. In aspects, the data acquisition layer may include additional software for performing process modeling such as chemometric modeling and/or other advanced process modeling, for example to process the raw data into product quality attribute information.

Also illustrated is a PAT instrument stack 704 including one or more analytic instruments and associated flow cells and probes. Exemplary flow cells that may be included are a Raman flow cell, a UV/VIS flow cell, a mid-IR flow cell, a near-IR flow cell, a DLS flow cell, and an index of refraction (IoR) flow cell, as described in more detail infra.

As illustrated, the PAT Box apparatus is in fluid communication via a recirculation loop 706 with, e.g., a unit operation of a pharmaceutical or biopharmaceutical manufacturing process, or an external vessel such as a process tank, a release tank, a hold tank, a mixing tank or a dilution tank, as described in more detail infra. The recirculation loop 706 may be connected via one or more valves to one or more of a material recycle flow path, a waste flow path, and a forward processing flow path.

FIG. 7B schematically illustrates an embodiment where two PAT Box apparatuses are connected via an electronic feedback loop to a PAT Control Cabinet where the HMI is configured for two-way communication with the instruments of the PAT instrument stack. As depicted here, each set of analytic instruments is in fluid communication via a recirculation loop with a unit operation, which may be e.g., a bioreactor, a chromatography apparatus, a filtration apparatus, etc., or a process vessel, which may be e.g., a hold tank, a mixing tank, etc.

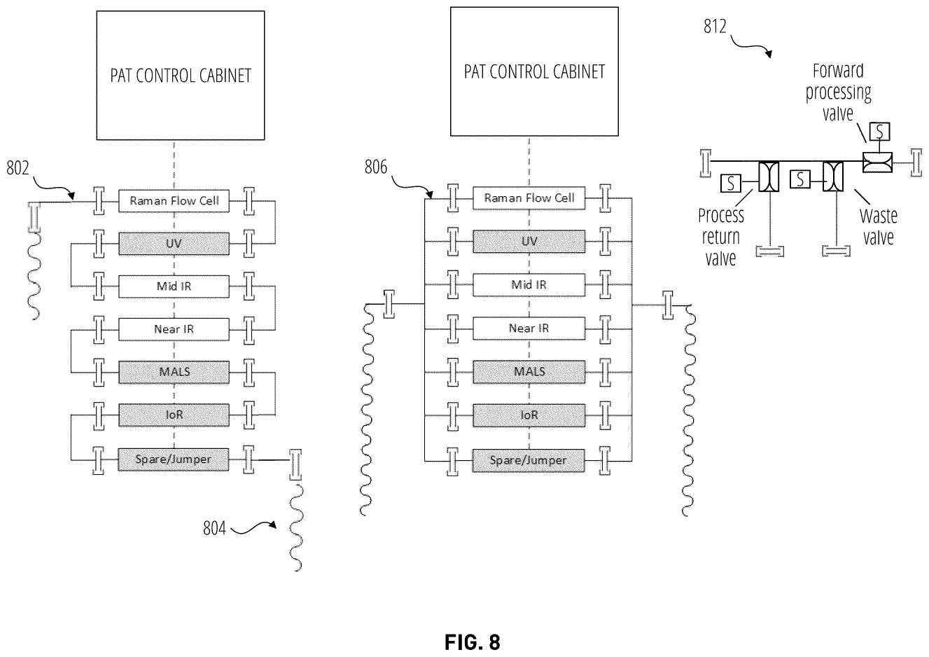

FIG. 8 is a schematic illustration of the flow cells of the PAT instrument stack. The flow cells may be arranged either in series (left) or in parallel (right) in relation to the flow path, depending on the needs of the end-user. For example, flow cells connected in series allow for a “plug and play” configuration in which additional flow cells may be added end to end on the string of PAT flow cells, as needed. This configuration occupies a larger footprint compared to flow cells configured in parallel. Flow cells configured in parallel occupy a smaller footprint, but are somewhat less flexible than a series configuration because flow cell tubesets and/or piping need to be preconfigured.

The schematic illustrates exemplary flow cells that may be included in a flow kit. These include a Raman flow cell, a UV/VIS flow cell, a mid-IR flow cell, a near-IR flow cell, a multi-angle light scattering (MALS) instrument flow cell, and an index of refraction (IoR) flow cell. In an aspect, the UV/VIS flow cell is a variable pathlength flow cell such as a FlowVPX® flow cell available from Repligen Corp.

The provision of customized flow kits suitable for different production modalities highlights the versatility of the PAT Box apparatus. Different production modalities, such as RNA, DNA, monoclonal antibody, viral vector, etc., may require different technologies to measure relevant product criteria, including product quality attributes. When comparing testing methods between modalities, there are some technologies that measure product attributes for multiple modalities and some than can only measure product attributes for a single modality. For example, some forms of spectroscopy can measure both RNA concentration and protein concentration. However, a MALS device may be able to measure lipid nanoparticle size for an RNA production line, but it may not be able to measure any product attributes for a monoclonal antibody process.

This problem is addressed by the provision of a plurality of modular flow kits, each adapted to a production modality. The modular flow kits are adapted for ease of replacement such that one may be easily removed and replaced with another. This allows for a single PAT Box apparatus to be utilized for multiple different modalities of production. For example, where the modality is RNA production, the flow kit may include RNA specific flow cells such as Mid IR, Near IR, and MALS. In another example, where the modality is antibody production, the flow kit may include protein specific flow cells such as IoR, DLS, FT-IR, and fluorescence. However, applications of the PAT skid are not intended to be limited to the RNA gene therapy bioprocess modality. The PAT skid can be applied to other modalities in the pharmaceutical manufacturing industry as well as other industries such as, but not limited to, food, purified water, and buffer solutions manufacturing.

Accordingly, in aspects, a flow cell tubeset may be provided as a flow kit for installation in a PAT Box with in-line process analytical instruments in a series or parallel flow path configuration along with associated tubing, valves, and connectors. In one aspect is provided a single use flow kit where the flow cells are arranged either in series or in parallel. For example, in FIG. 8, the flow cells in white, Raman, MidIR, and NearIR, may be supplied as a single use flow kit including single-use tubing, connectors, and flow cell components. Not all components may be single use and may need to be removed from the flow kit after use for cleaning and/or sanitization.

In another aspect, provided is a single use flow kit with smart waste and release valves where the flow cells are arranged in either a series or parallel configuration and the kit includes associated tubing, valves, and connectors. In accordance with this aspect, provided are multiple outlets connecting to single use “smart” pinch valves for waste and forward processing. In an aspect, single use tubing is placed into permanent pinch valves for use during processing. Once the batch is complete, the flow kit is removed from the holders (not shown) and pinch valves are discarded. In an aspect, the pinch valves may be automated solenoid pinch valves.

In another aspect, provided is a single use flow kit with smart return, waste and release valves where the flow cells are arranged in either a series or parallel configuration and the kit includes associated tubing, valves, and connectors. In accordance with this aspect, provided are multiple outlets connecting to single use “smart” pinch valves for process return material, waste, and forward processing. The single use tubing is placed into permanent pinch valves for use during processing. Once the batch is complete, the flow kit is removed from the holders (not shown) and pinch valves and discarded.

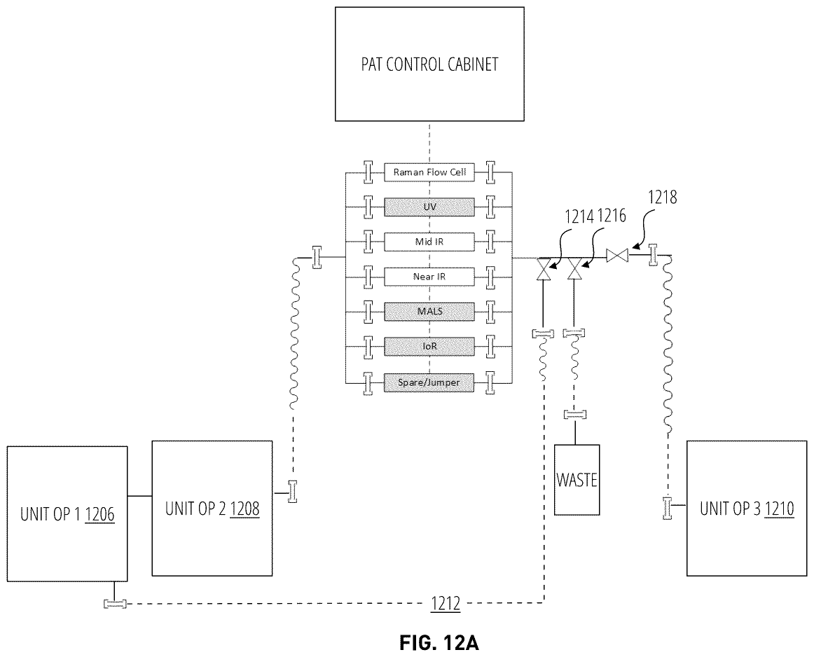

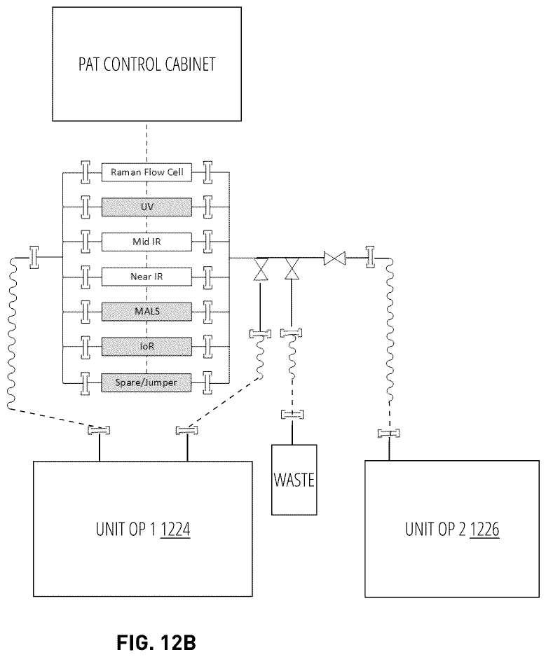

Shown as an inset in FIG. 8 is an exemplary configuration of a smart valve set 812 including a process return valve, a waste valve, and a forward processing valve. In aspects, these valves may be automated solenoid pinch valves. See FIG. 12A and FIG. 12B for exemplary configurations of such a valve set.

The flow cells of the PAT instrument stack may be fluidly connected to one or more unit operations and/or vessels via suitable connectors 806 and tubing 804. In aspects, the tubing 804 may be flexible tubing. The flow cells and flexible tubing can be connected to each other and unit operations via any suitable connector. Suitable connectors include, for example, sanitary tri-clamp connectors, hose barb connectors, tube compression connectors, quick connects, aseptic connectors, or other standard connectors used in the biopharmaceutical industry.

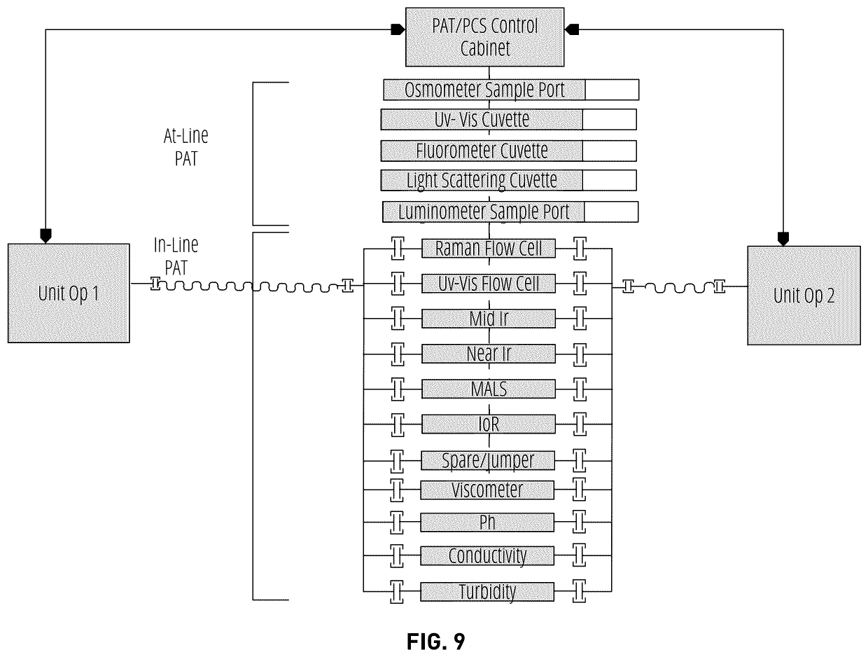

FIG. 9 is a schematic illustrating an aspect of a PAT Box Apparatus wherein the PAT instrument stack is adapted for both at-line and in-line process analytic instruments. As shown, the PAT instrument stack includes sample ports and/or cuvettes adapted for at-line data acquisition and material analysis, as well as flow cells for in-line data acquisition and material analysis. Exemplary at-line PAT instruments may include an osmometer, a UV-VIS spectrophotometer, a fluorometer, a light scattering instrument, and a luminometer. In operation, an automated system or operator may obtain a sample from a unit operation or vessel and transfer it to the cuvette and/or sample port of the at-line PAT instrument for rapid data acquisition and material analysis on the manufacturing floor.

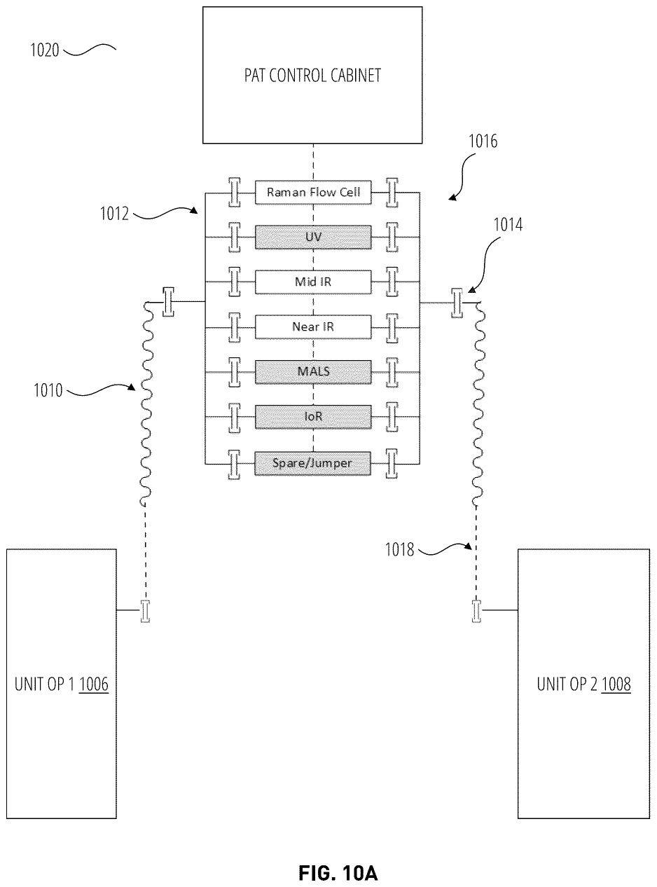

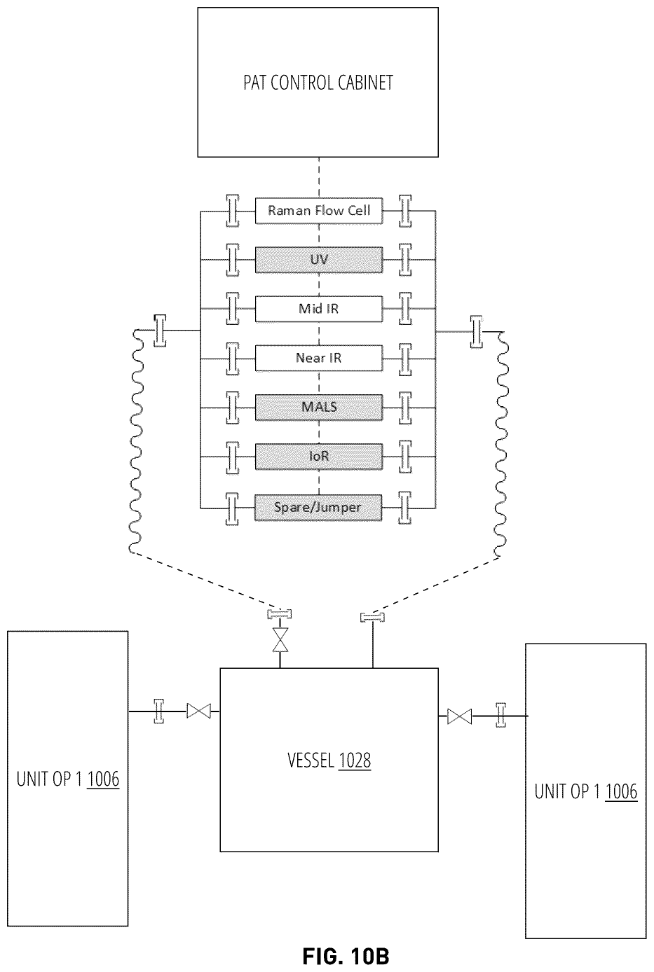

FIG. 10A is a schematic illustrating an embodiment of a PAT Box apparatus where the PAT Box is integrated in-line between two unit operations of a pharmaceutical or biopharmaceutical manufacturing process. The schematic depicts a PAT Box apparatus situated inline between a first unit operation 1006 and a second unit operation 1008. By way of example the first unit operation may be a bioreactor unit, e.g., for culturing cells or performing an in vitro transcription (IVT) reaction, and the second unit operation may be a single pass TFF unit. Throughout this disclosure, exemplary unit operations are depicted for purposes of illustration only and are not intended to be limiting.

As illustrated in FIG. 10A, the PAT instrument stack comprises six flow cells and a spare in a parallel configuration. The PAT instrument stack is fluidly connected to the unit operations, for example, via a connector 1014 and flexible tubing 1010, as discussed above in relation to FIG. 8. The PAT Box apparatus may also include a pump for recirculating process fluid from a first unit operation through the flow cells of the PAT instrument stack and to the second unit operation, or optionally to an external vessel, or to a recycle line back to an upstream unit operation, as discussed in more detail below.

In aspects, this configuration may also include an optional vessel (not shown) external to the PAT Box apparatus housing. In aspects, the optional vessel is included in-line between a unit operation and the associated PAT Box. In aspects, the vessel is a hold vessel, a release tank, a mixing tank, a dilution tank, or a stirred tank reactor (STR). The vessel may include a plurality of ports, including IO ports for data transfer between the vessel and the PAT Box. In aspects, the vessel incorporates one or more analytical detectors, sensors and/or probes which may include one or more of an index of refraction (IoR) sensor, a conductivity sensor, a temperature sensor, pH probe, pressure sensor, turbidity sensor, oxidation-reduction potential sensor, dissolved oxygen probe, ozone sensor probe, an on-line photometer to measure silica and phosphate, a Microfluidic capillary electrophoresis analyzer to measure chloride and sulfate, and/or a probe sensor to measure nitrate.

In aspects where an external vessel is not included, the configuration may also include one or more sensors, e.g., IoR sensors or other sensors, or one or more analytical detectors and/or probes at one or more of the unit operations.

In accordance with any of the foregoing aspects, the one or more analytical detectors, sensors and/or probes may include a pH sensor, conductivity sensor, temperature sensor, pressure sensor, turbidity sensor, oxidation-reduction potential sensor, dissolved oxygen probe, an ozone sensor probe, an on-line photometer e.g., to measure silica and phosphate, a Microfluidic capillary electrophoresis analyzer, e.g., to measure chloride and sulfate, and a probe sensor to measure nitrate. Exemplary sensors may be depicted for purposes of illustration in various figures of the present disclosure and such exemplary illustrations are not intended to be limiting.

In aspects, this configuration may also include valves and pumps are configured to control the flow of process fluid between each unit operation and the PAT instrument stack. Operation of one or more valves and/or pumps may be controlled by a controller of the PAT Control Cabinet.

FIG. 10B is a schematic illustrating an embodiment where a PAT Box apparatus is connected via a vessel 1028 situated between two unit operations of a pharmaceutical or biopharmaceutical manufacturing process. The vessel is external to the PAT Box apparatus housing and may be a hold vessel, a release tank, a dilution tank, etc. The vessel may include a plurality of ports, including IO ports for data transfer between the vessel and the PAT Box apparatus. In aspects, the vessel incorporates one or more analytical detectors, sensors and/or probes including for example, a conductivity sensor, a temperature sensor, and/or pH probe (not shown). Such probes may be connected integrally into the vessel.

Also illustrated is the PAT instrument stack where the flow cells are integrated in parallel in the flow path, as discussed above in relation to FIG. 10A. In operation, process fluid moves in a recirculation scheme from the vessel through the instrument stack and back into the vessel via operation of a pump (not shown). Fluid flow may be controlled via the coordinated operation of one or more pumps and valves. For example, suitable valves and pumps may be configured to control the flow of process fluid between (i) the upstream or first unit operation and the vessel; (ii) the vessel and the flow cells of the PAT instrument stack; and (iii) the vessel and the downstream or second unit operation. This configuration may be useful, for example, for real time release testing between unit operations in a continuous process.

Alternatively, the PAT Box may be connected to a single unit operation. An example of this use case is a unit operation that involves a biological reaction such as IVT where it would be advantageous to monitor the reaction kinetics for characterization and optimization.

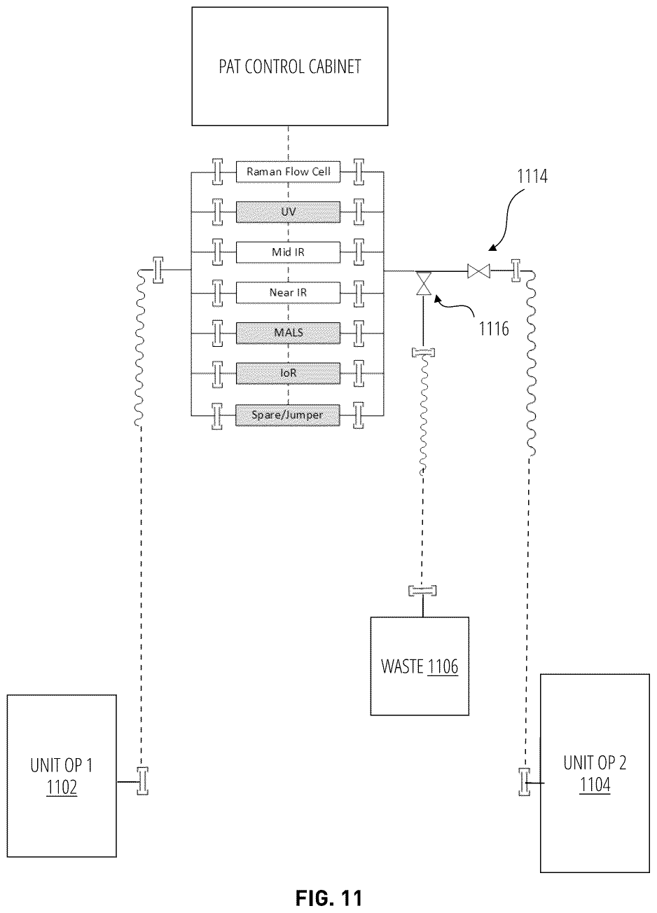

FIG. 11 is a schematic illustrating an embodiment of a PAT Box apparatus where the PAT Box is integrated in-line between two unit operations as depicted in FIG. 10A above, but with the addition of two smart valves, a waste valve and a forward processing valve. In this context, a “smart valve” refers to a valve that can be remotely and automatically configured to an open or closed state when certain process or analytical conditions are met by actuation of control software. Such smart valves are commercially available, for example, one option for a “smart” valve is a pneumatically operated diaphragm valve manufactured by GEMU, or the GEMU electrically operated single use pinch valve. In operation, the forward processing valve remains closed when in-process testing criteria measured by the PAT flow cells is not met. Where the criteria are satisfied, the forward processing valve opens and process fluid is allowed to flow to the next unit operation (Unit Operation 2). The waste valve may be opened or remains open when in-process testing criteria measured by the PAT flow cells is not met. Where the criteria are satisfied, the waste valve closes.

FIG. 12A is a schematic illustrating a configuration where the PAT Box is integrated in-line between two unit operations of a manufacturing process, depicted as 1208 and 1210. This configuration may be used, for example, for real time release testing at Unit Op 2 1208. In operation, process fluid from a first unit operation 1206, for example a bioreactor or IVT reaction vessel, flows to a second unit operation 1208 and then passes into the flow cells of the PAT instrument stack, depicted here in a parallel configuration with respect to the flow path. Three smart valves are positioned downstream of the instrument stack, each operating to open or close a fluid conduit connected to a unit operation for recycling 1214, a waste container 1216, or the next unit operation in the manufacturing process 618. As illustrated, the material recycle valve 1214 controls access to a reagent recovery loop 1212 in fluid communication with the first unit operation 1206. This configuration may be used, for example, to recover and/or recycle reagents back to the first unit operation. The waste valve 1216 controls access to a waste conduit in fluid communication with a waste vessel. The forward processing valve 1218 controls access to a conduit in fluid communication with the downstream unit of operation 1210. In operation, each of the smart valves is actuated in accordance with predetermined in-process criteria as measured by the PAT flow cells.

FIG. 12B illustrates a configuration where the PAT Box is integrated at a first unit operation, Unit Op 2 1208. In operation, process fluid recirculates through the instrument flow cells and may be recycled back into the unit 1224 via the recycle valve. Where process criteria are met, the recycle valve closes, the waste valve remains closed, and the forward processing valve is opened, allowing the process material to pass to the next unit operation, 1226. If the criteria are not met or deemed to be sub-standard, the waste valve may be opened to send process fluid to a waste container.

To further illustrate this configuration by way of an example, the first unit operation may be a single pass TFF and the process fluid may be recycled through the TFF until a predetermined concentration condition is met. At the point, the process fluid would be allowed to pass to the next unit operation. Otherwise, the process material could be allowed to go to waste.

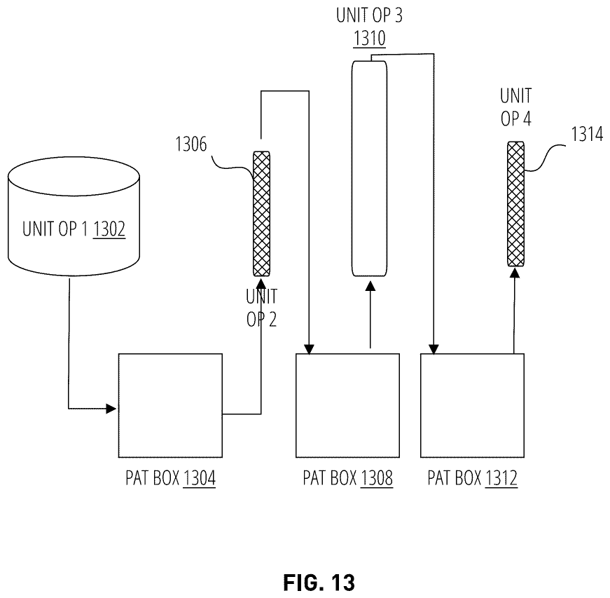

FIG. 13 is a schematic illustrating a configuration where a plurality of PAT Box apparatuses are integrated inline between subsequent unit operations of a pharmaceutical or biopharmaceutical manufacturing process. The schematic depicts a system including a first inline PAT Box 1304 situated inline between a first unit operation 1302, which may be, for example, a bioreactor or IVT reaction vessel, and a second unit operation 1306, which may be, for example, a tangential flow filtration (TFF) module. A second PAT Box 1308 is situated inline between the second unit operation 1306 and a third unit operation 1310, which may be, for example, a chromatography module. A third PAT Box 1312 is situated inline between the third unit operation 1310 and a forth unit operation 1314, which may be, for example, a second TFF module. In operation, each PAT Box is configured with a set of flow cells and instruments to measure process parameters including CPPs and/or CQAs, compare the measured parameters against predetermined criteria, and control the flow of process fluid from one unit operation to the next, for example via control of smart valves as discussed above. Accordingly, this configuration may also include valves and pumps configured to control the flow of process fluid between each unit operation and the PAT Box, as detailed above in relation to FIG. 12A and FIG. 12B.

The versatility of the PAT Box apparatus is such that it may be adapted for monitoring and control of diverse unit operations, including a biological reaction operation, such as IVT, a filtration operation, a chromatography operation, a lipid nanoparticle (LNP) multi-phase mixing operation, and/or an excipient addition operation. Other unit operations may include a fermentation operation, a cell lysis operation, a protein capture operation, a viral inactivation/filtration (VI) operation, an enzymatic reaction operation, a fill/finish operation, and/or a polishing operation.

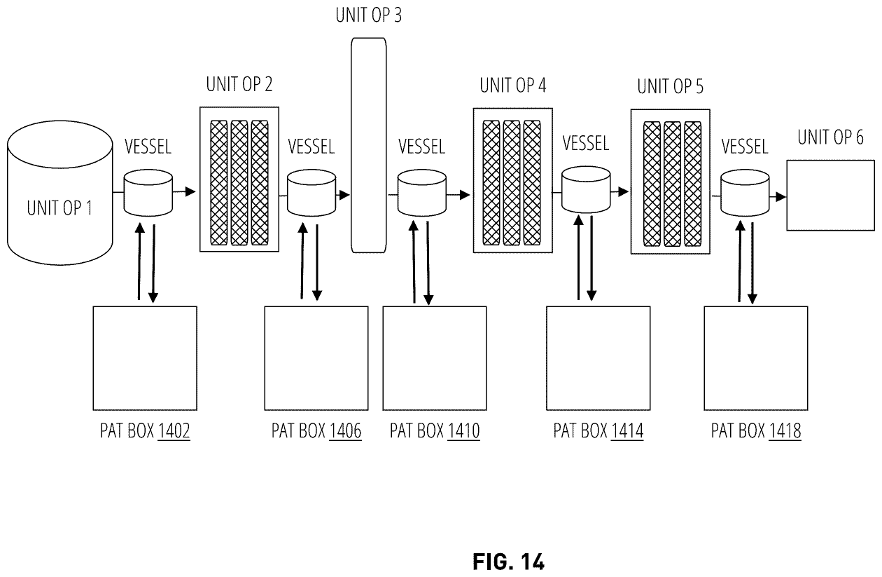

FIG. 14 is a schematic illustrating a configuration where several PAT Box apparatuses are integrated into a manufacturing process as in FIG. 13, but instead of being directly connected between each unit operation, the PAT Box apparatuses are each connected via an external vessel to a unit operation in the process. The vessels may be situated in-line between one or more of the unit operations and the associated PAT Box. In aspects, the vessel is a hold vessel, a release tank, a dilution tank, or a stirred tank reactor. The vessel may include a plurality of ports, including IO ports for data transfer between the vessel and the PAT Box. In aspects, the vessel incorporates one or more analytical detectors, sensors and/or probes including for example, a conductivity sensor, a temperature sensor, and/or pH probe.

In operation, a first PAT Box 1402 monitors and optionally controls a process within a first unit operation (Unit Op 1). A second PAT Box 1406 monitors and optionally controls a second unit operation (Unit Op 2). Subsequent PAT Boxes, 1410, 1414, and 1418 monitor and optionally control other downstream units of operation. In aspects, this configuration may also include valves, including smart valves, and pumps configured to control the flow of process fluid between each unit operation, the external vessel and the PAT Box. The PAT Box apparatuses are configured for easy integration at key points in the manufacturing process, for example via the use of standard connectors such as tri-clamps, quick connects, etc., to connect the vessels to the unit operations and to the PAT Boxes.

This configuration may be useful, for example, during process development to identify CCPs for monitoring and control. This configuration may also be useful for real time release and/or verifying in-process control measurements are within limit criteria for forward processing.

In aspects, the flow path may be configured such that the process fluid from a given unit operation recirculates through the flow cells of the in-line PAT Box and returns to the same unit operation, for example as illustrated in connection with FIG. 12B. Alternatively or in addition, the flow path may contain one or more “smart” valves configured to control the flow of process fluid back to a unit operation for recycling, or to a waste container, or forward to the next unit operation in the manufacturing process, depending on whether or not predetermined criteria are satisfied, for example as illustrated in FIG. 12A and FIG. 12B.

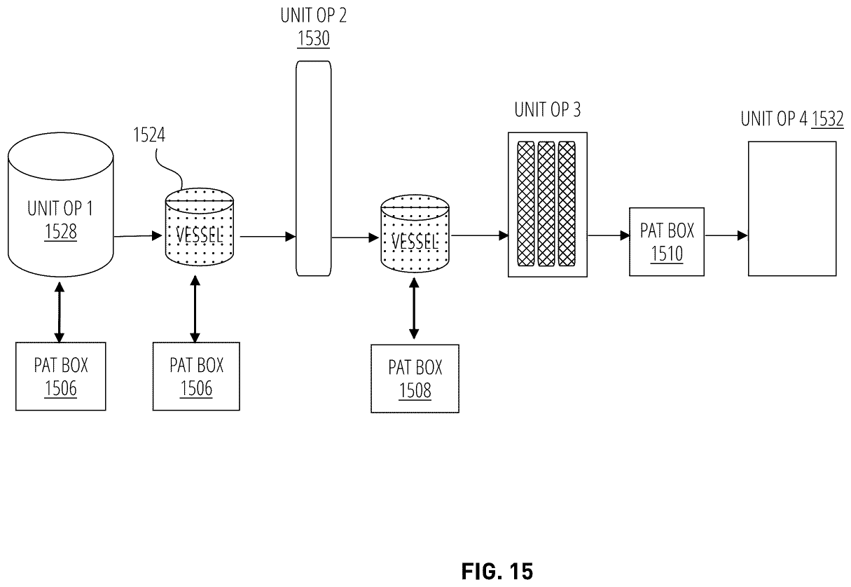

FIG. 15 is a schematic illustrating a configuration of a manufacturing process where a plurality of PAT Box apparatuses are integrated in different ways. As depicted here, the manufacturing process may be made continuous or integrated with surge or hold vessels configured between the unit operations. PAT Boxes may be integrated and connected to these vessels to monitor and control the process. PAT Boxes may also be placed in-line between unit operations or integrated directly into a unit operation, for example to monitor an IVT reaction, a cell culture, or fermentation. With reference to FIG. 15, a first PAT Box 1506 is configured to recirculate process fluid from a first unit operation 1428 and return the process fluid to the same unit operation. The next two PAT Boxes 1506 and 1508 are configured to connect to an external vessel 1524 which is in-line between two unit operations in the process. The external vessel 1524 may comprise a body in the form of a flexible or a rigid container defining an interior space, inlet and outlet ports, an optional impeller for fluid recirculation, and an optional probe or sensor. The external vessel may be a hold vessel, a release tank, a dilution tank, or a stirred tank reactor and may include one or more of an IO port for data transfer and one or more analytical detectors, sensors and/or probes, as described above in connection with FIG. 14. A third PAT Box 1510 is situated in-line between the last two unit operations in the process, for example as described above in connection with FIG. 13.

It is understood that here also the flow path may be configured such that the process fluid from a given unit operation recirculates through the flow cells of the in-line PAT Box and returns to the same unit operation, and/or the flow path may contain one or more “smart” valves configured to control the flow of process fluid back to a unit operation for recycling, or to the waste container, or forward to the next unit operation in the manufacturing process, depending on whether or not predetermined criteria are satisfied, as discussed above.

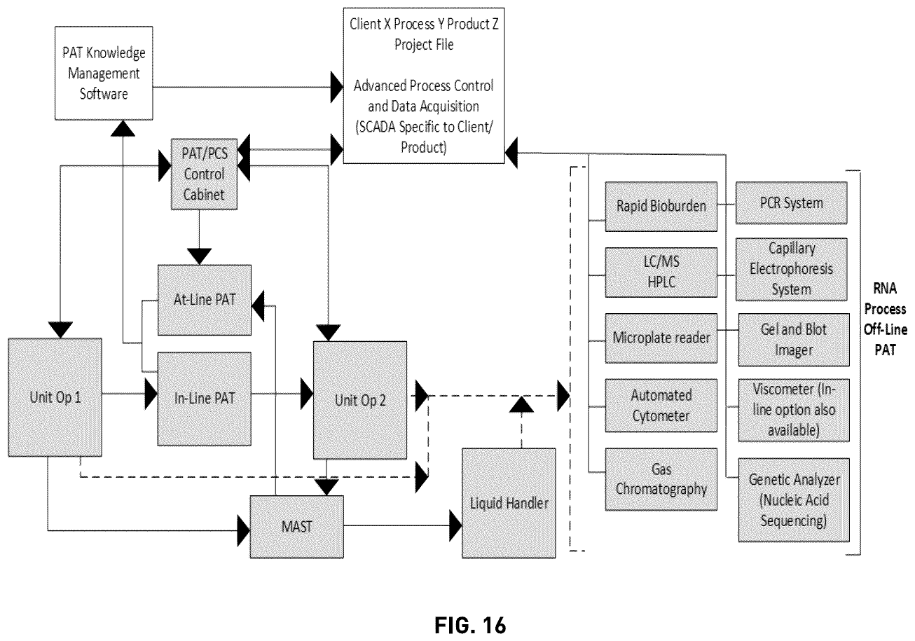

FIG. 16 is a schematic illustrating integration of a PAT Box apparatus including in-line and at-line analytic instruments with unit operations, off-line instruments, process specific SCADA and a project file data. In operation, the PAT Control Cabinet, e.g., via its APC and SCADA, receives data from the PAT instrument stack which is connected to the in-line and/or at-line instruments, shown for illustration purposes as situated between two unit operations of a process. The PAT Control Cabinet processes the data and may also be configured to send commands to the at-line and in-line analytic instruments via the PAT Knowledge Management software and SCADA.

Data from off-line analytic instruments may be incorporated via automatic or manual sampling and transfer (dashed line) of process fluid to the off-line instruments. Raw data from off-line analytics is transferred to a project file which is accessed by the PAT Knowledge Management software.

Illustrated are non-limiting examples of off-line process analytic instruments including a rapid bioburden instruments, LC-MS and HPLC instruments, a microplate reader, an automated flow cytometer, a gas chromatograph, a polymerase chain reaction (PCR) system, a capillary electrophoresis system, a gel and/or blot image system, a viscometer, and a nucleic acid sequencer.

A system comprising one or more PAT Box apparatuses and associated digital architecture provides a universal solution to fully integrate any pharmaceutical or biopharmaceutical manufacturing process with a wide range of process analytic tools. In this context, the term “digital architecture” refers to the design and integration of hardware, software, networks, and data structures. For example, the digital architecture includes servers that store process data, analytic data, and offline data, the software that enables interactions between the data, and the protocols that govern how data is shared between system components. In aspects, the digital infrastructure may include a Knowledge Hub which may include process specific data, including historical process run and analytical data, design of experiments (DOE) and process development and design space data, as well as clinical data. In aspects, user data may be fed into the Knowledge Hub by any one or more of import algorithms on csv and/or propriety data, manual entry, online portal and form, and/or Laboratory Information Management System (LIMS) facilitates sample integrity from off-line instruments and data transfer to Knowledge Hub.

Accordingly, in an aspect provided is a PAT Box apparatus configured to connect and send information to a Knowledge Hub, e.g., via PAT Knowledge Management software. In accordance with this aspects, the PAT Box apparatus may be configured to transmit data including spectrogram, chromatogram, and particle size density that may include over 3000+ points per second averaged over a specified period of time. In aspects, the data is converted into useful process parameters including e.g., concentration, particle size, purity, etc., for example by appropriate models. In aspects, the models may interface with a digital twin to model critical quality parameters and critical process parameters. The digital twin models may include mechanistic, data-driven, machine learning, and hybrid models that incorporate different physics and phenomena in the process. The digital twin may also interface with machine learning and optimization algorithms that optimize the process for performance, cost, and yield. The Knowledge Hub may also transmit adjustments or process recipes directly to the control system, e.g., by sending command bits to PAT instruments and smart valves for monitoring, open/close etc. Human operators at the control system may acknowledge or dismiss based on the floor priorities. The control system may also runs through system checks to acknowledge new parameters are within a control range.

A prototype PAT Box Apparatus was constructed which included the following instruments:
1. Raman Instrument (MarqMetrix AIO)
2. UV VIS Instrument (Repligen CTech FlowVPX)
3. Mid-IR Instrument (IRUBIS)
4. NIR Instrument (Avantes)
5. Multi Angle Light Scattering (MALS) Instrument (Waters)
6. DLS Instrument (nanoparticle size analyzer, INPROCESS NanoFlowSizer)

The instruments where housed on a custom skid which also include power, communications, and pump components. Data was collected to train models for prediction of CQAs in mRNA drug substance (DS) and drug product (DP) unit operations. The following Figures and associated descriptions highlight the functions of the PAT Box Apparatus operation in practice.

### Example 1—Using Raman to Measure mRNA and NTP Concentration at IVT Unit Operations

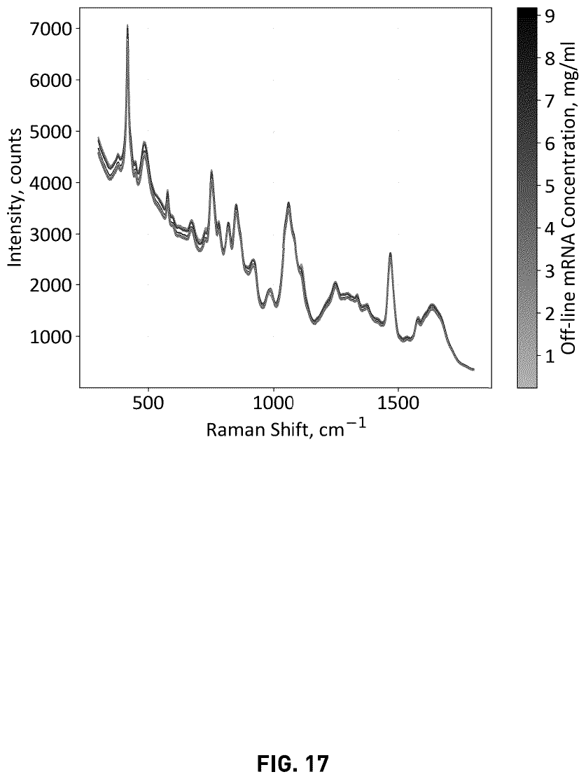

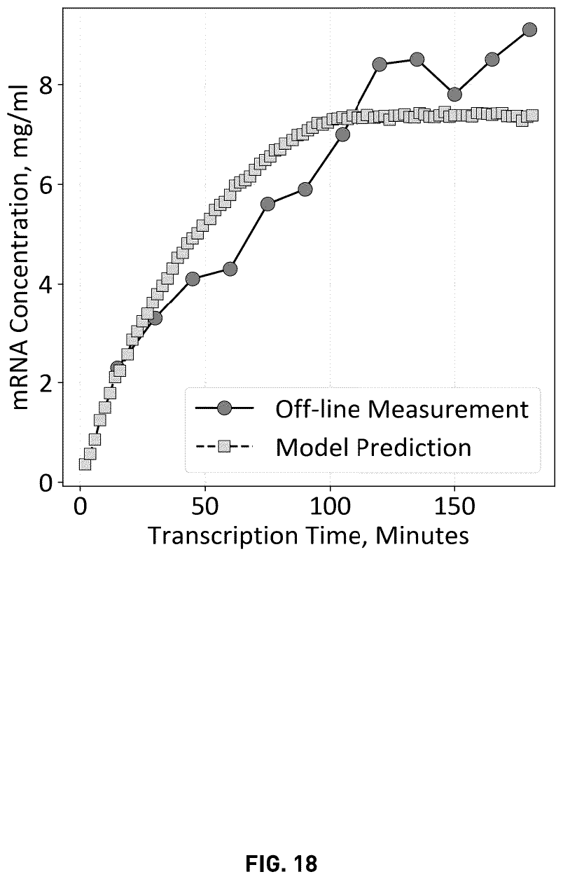

A PLS model was trained using in-line Raman data collected during multiple IVT runs and calibrated using off-line UV-VIS measurements. This model was then used to predict mRNA concentration during additional IVT runs by feeding Raman data to the model in real-time. The Raman instrument collected one measurement per two minutes. The data was fed into the PLS model in real-time and model predictions were generated in real-time. Additional off-line samples were collected during the run to compare with the model predictions. FIG. 17 is a graph illustrating raw spectra collected from an in-line Raman flow cell collected during an IVT test operation. The PLS model predictions for mRNA concentration over time are shown in FIG. 18 as compared to off-line UV-VIS measurements for the IVT test operation.

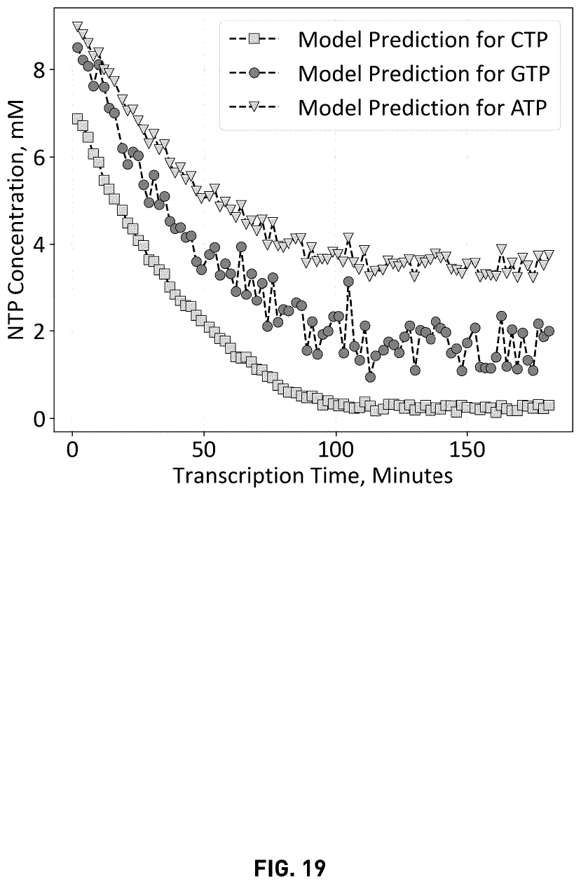

FIG. 19 is a graph showing PLS model predictions of NTP concentration (mM) versus transcription time (min) for the IVT test operation based on Raman spectroscopy measurements. Predicted CTP, GTP, and ATP concentrations are shown by squares, circles and triangles, respectively.

The use of in-line Raman technology for measuring critical quality attributes (CQA's) such as NTP concentration as illustrated in this example represents an improved method for several reasons. For example, as an in-line analytic, it obviates the need for manual sampling and allows for real-time data acquisition. The models developed are also used in real-time to monitor the process CQAs thereby providing instantaneous information about process performance. In-line Raman technology as described herein represents an alternative to off-line analytical methods such as UV or HPLC which may take hours to obtain the information required to move a process forward to the next step of the production. During off-line testing a process cannot move forward until results are verified, presenting a bottleneck in analytics and process holding step(s). There is also lack of real-time acquisition in manual operation as samples are taken out by an operator rather than automated and measured via technology in place, such as Raman.

This example demonstrates in-line Raman flow cell measurement of an in vitro transcription unit operation. The flow cell measurement enabled implementation of Raman measurement to various scales from 10 ml-250 ml bioreactors, making this technology scale agonistic. In order to adapt Raman analytics to in-line continuous measurement, it was necessary to overcome engineering challenges such as maintaining the process temperature. This was accomplished using insulation and a shorter re-circulation tube combined with increasing the flow rate in the re-circulation loop. At the same time, Raman acquisition parameters were optimized to achieve enough spectral quality to distinguish between fingerprint of different process components.

### Example 2—Using in-Line UV-VIS to Measure mRNA Concentration at Downstream Unit Operations

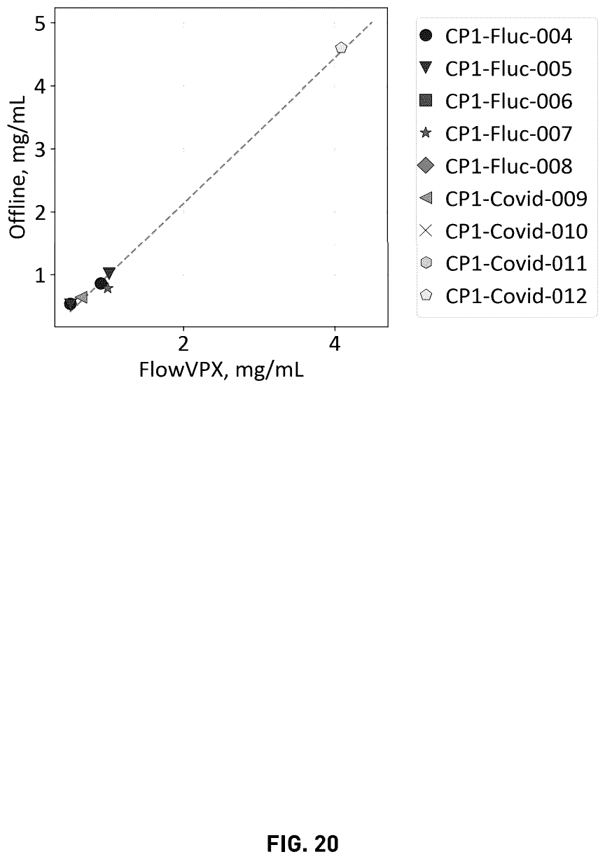

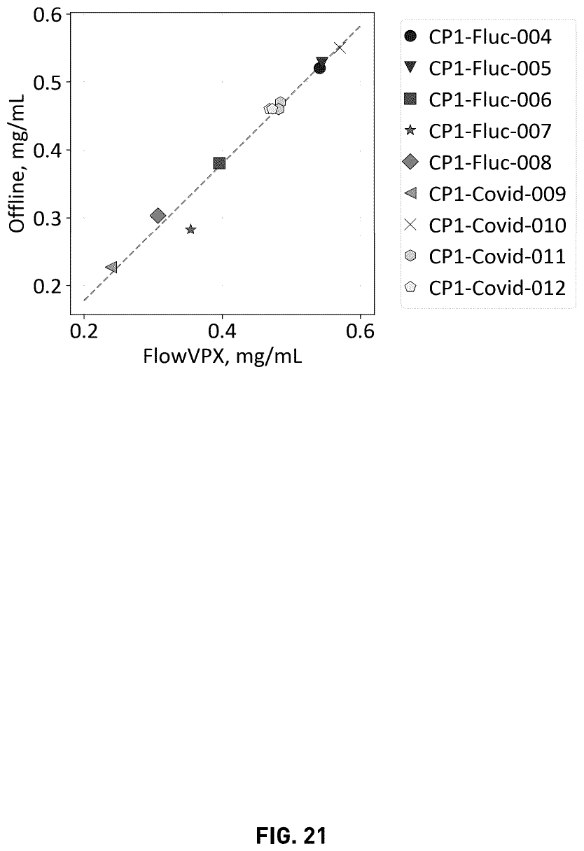

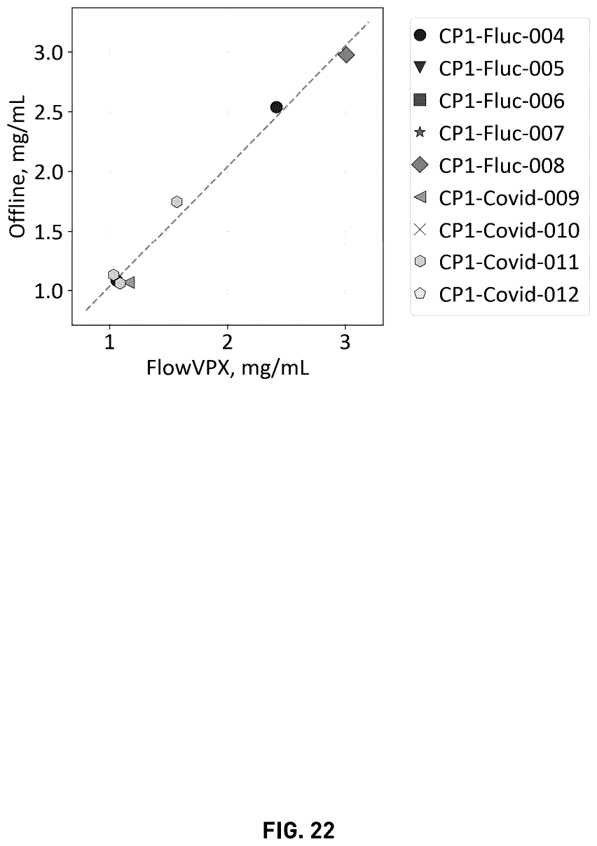

In this example of a continuous process, the IVT unit operation is followed by a first tangential flow filtration (TFF1) operation, an oligo-dT affinity chromatography (OdT) operation, and a second tangential flow filtration (TFF2) operation. UV-VIS instruments were used in an in-line flow cell setup to measure mRNA concentration in real-time at three points in the continuous process, at a TFF1 pool, a OdT pool, and a TFF2 pool. The results of these measurements were compared to off-line UV-VIS measurements for validation purposes. FIG. 20 illustrates the correlation between in-line UV-VIS mRNA concentration measurements (mg/ml) and off-line UV-VIS measurements at the TFF1 pool. The R² is equal to 0.99. The RMSE is equal to 0.19 mg/mL. FIG. 21 illustrates the correlation between in-line UV-VIS mRNA concentration measurements (mg/ml) and off-line UV-VIS measurements at the OdT pool. The R² is equal to 0.97. The RMSE is equal to 0.02 mg/mL. FIG. 22 illustrates the correlation between in-line UV-VIS mRNA concentration measurements (mg/ml) and off-line UV-VIS measurements at the TFF2 pool. The R² is equal to 0.98. The RMSE is equal to 0.04 mg/mL.

These results demonstrate the feasibility of using in-line UV-VIS analytics to measure mRNA concentration during a continuous mRNA manufacturing process, avoiding the need for process holds at critical points as required for off-line analytical methods. Real-time monitoring of mRNA concentration as demonstrated here also enables data-driven decisions to support faster and more effective production of the therapeutic. These methods are also useful to predict at which point in production a problem may be occuring due to the ability to see in real time where product specifications are not being met.

### Example 3—USING Mid-IR to Measure mRNA Concentration at IVT Unit Operation

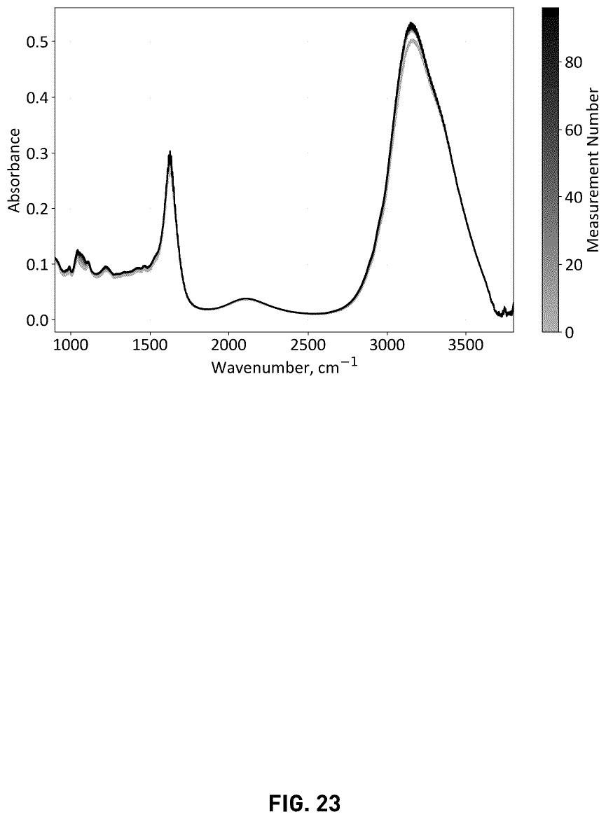

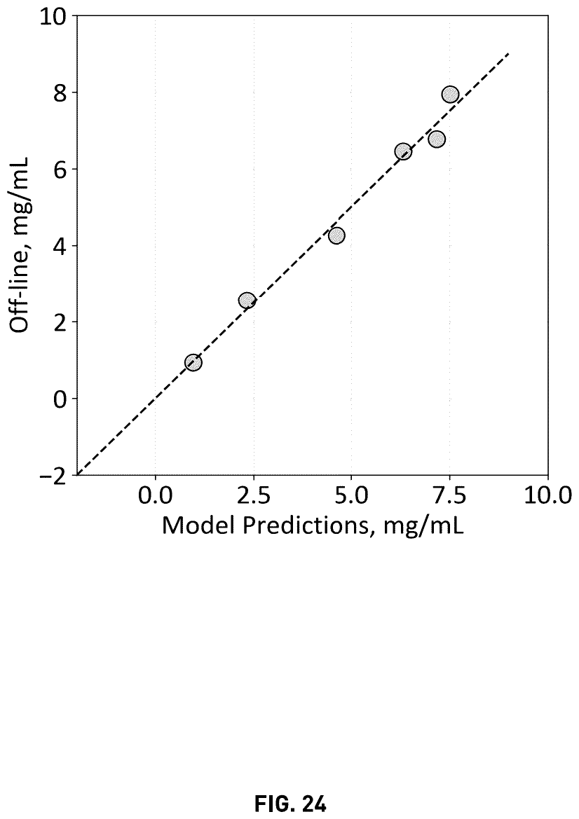

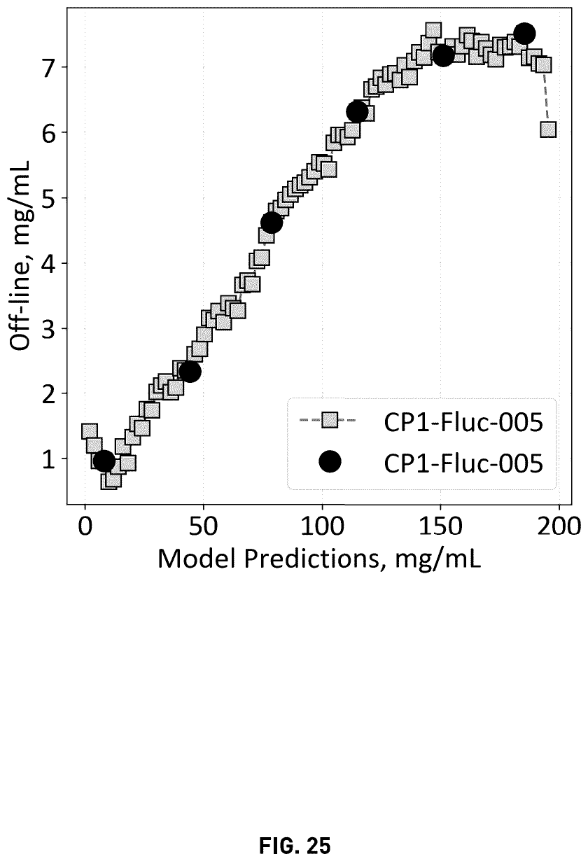

In this example, a PLS model was trained using in-line Mid-IR data collected during an IVT run CP1-Fluc-005 and calibrated using off-line UV-VIS measurements. FIG. 23 illustrates raw spectra collected from in-line Mid-IR flow cell in the IVT run. FIG. 24 illustrates a PLS model training plot showing alignment of off-line UV-VIS measurements to the PLS model predictions. The R² is equal to 0.99. The RMSE is equal to 0.3 mg/mL. FIG. 25 illustrates PLS model prediction (squares) of all measurements taken during the IVT run compared to off-line UV-VIS measurements (circles).

Similar to Raman and UV, midIR technology allows real-time integration of the system into a process workflow. Sample during production is measured in real-time and information is then obtained to make a decision if enough of the product or CQA of interest has been made and is within specifications. This in-line method is an alternative to off-line testing such as UV or HPLC reducing time and providing a more efficient process.

The present example describes real-time mid-IR technology deployed in mRNA manufacturing and in IVT and Drug Product unit operations using flow cell point of measurement. A mid-IR flow cell was engineered to be in series with Raman and UV-VIS flow cells in order to minimize the amount of sampling from unit operations. The use of flow cells is crucial to the operation of the PAT Box apparatus as each flow cell can be removed or added depending on the analytical needs of each unit operation, thereby providing modular capability of the apparatus.

For Mid-IR, optimization of the sequence of operations had to be resolved. Mid-IR require background spectral subtraction to highlight changes in the spectra. Selection of the background spectra according to the sequence of material addition to IVT was studied to improve Mid-IR post processing and modeling accuracy. The Mid IR flow cell needed to be installed in series with the Raman and UV-VIS flow cells to minimize the flow path length and minimize tubing length between flow cells thus minimizing hold up volume. As hold up volume increases, temperature differential between the external heat source (for example heat plate or vessel jacket) for the bioreactor and the internal reaction temperature due to the increased heat transfer lost through the recirculation tubing and thus increased the load on the external heat source. Increased hold up volume was also correlated to slower reaction kinetics in the reaction vessel. Therefore, it was imperative to maintain the shortest recirculation loop possible.

### Example 4—Waters Multi Angle Light Scattering (MALS) Instrument

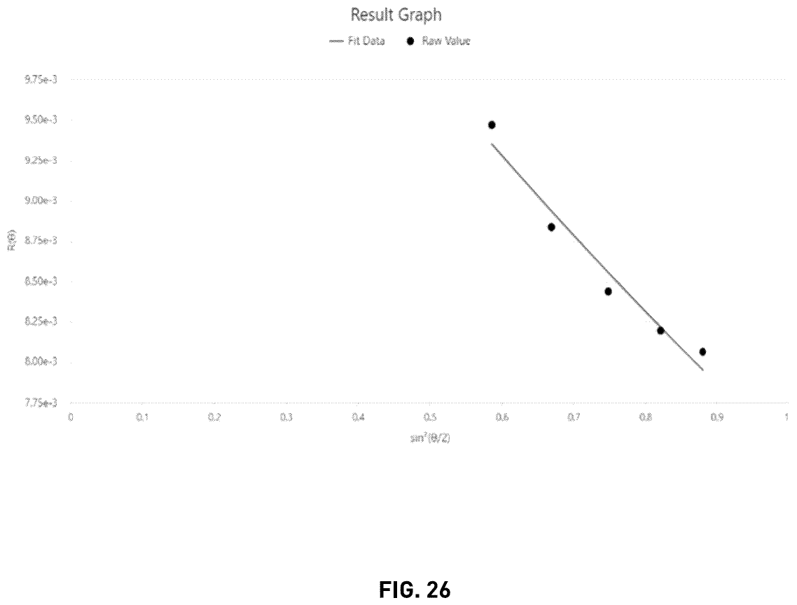

In this example a MALS system is used to characterize lipid nanoparticles post LNP production. A sample was pulled from the LNP pool. FIG. 26 illustrates results from a Waters' MLAS system being used to obtain a correlation between the intensity of the scattered light and scattered light angle for the LNP samples.

The parameters for the MALS instrument were as follows:
7. Model—sphere
8. Particle RI Real—1.45
9. Particle RI Imaginary—0
10. Detectors—10, 12, 13, 14, 15, 16, 17, 18

The slice results indicated:
11. Slice index of 5.006
12. Particle concentration (1/mL) of 1.732E+11
13. Radius (nm) of 69.8

The region results indicated:
14. Region start—4.400
15. Region end—5.500
16. Average particle concentration (1/mL)—7.6E+9
17. Total number of particles—8.2E+10
18. Z—average radius (nm)—242

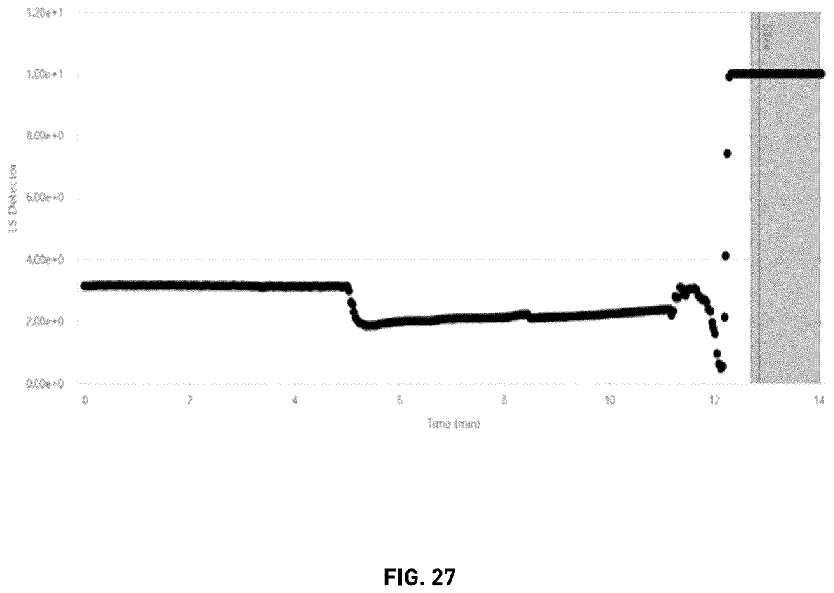

FIG. 27 illustrates a control graph of LS11 that shows detection of particles by the instrument vs time of the measurement. These results were obtained by off-line measurements as a proof of concept. MALS represents another analytic that can be integrated in-line with the manufacturing process. MALS can be utilized with data-driven models to determine important CQAs during the LNP process, such as size, Pdi, and concentration) to support real-time and continuous manufacturing of LNPs. Off-line methods used to measure similar CQAs require hours and sometimes days to obtain results. As with other off-line analytics, during this time the process is on hold until results are obtained, which ultimately slows down production. Another important aspect of in-line analytics as described here is that is allows to track the reaction process and identify any anomalies (if they occur) in real-time, having the ability to control and react quicker reducing the risk of batch loss.

### Example 5—In-Line DLS Instrument

This example characterized the ability of an in-line DLS instrument to measure CQAs relevant to lipid nanoparticles (LNP), including PDI and z-average in real-time (similar to MALS). Data for this step was gathered from a post LNP pool process step in the RNA manufacturing process.

Table 1 illustrates results from LSP NanoFlowSizer (in-line DLS):

| | Parameter | Value | Moving Average |
|---|---|---|---|
| 1 | Time (sec) | 10541.8 | |
| 2 | Number | 1766 | |
| 3 | Temperature | 25.7 | 25.7 |
| 4 | Z-average (nm) | 87 | 87 |
| 5 | Cumulant PDI | 0.33 | 0.33 |

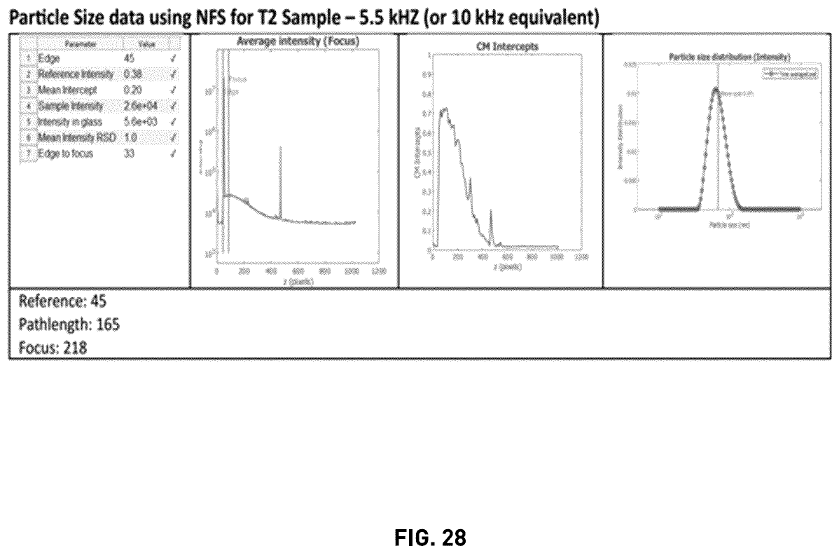

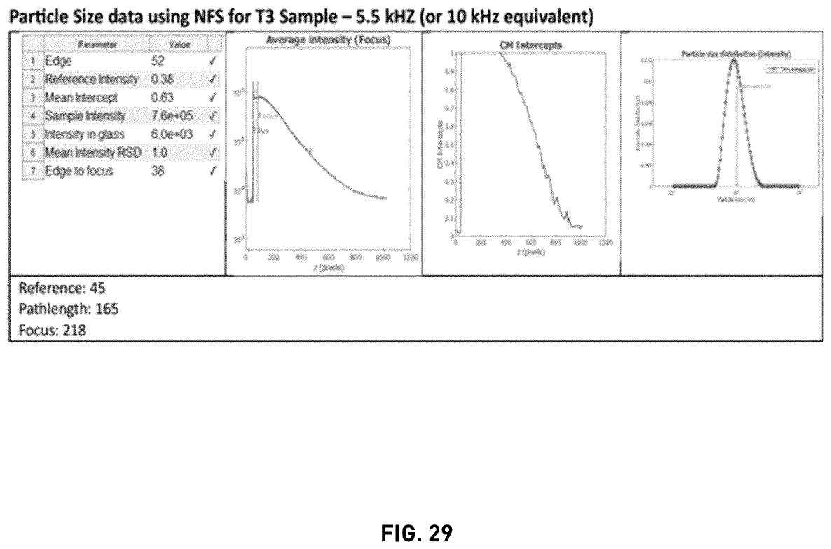

FIG. 28 is a graph of particle size versus time for the LNP sample as measured using a Z-average and polydispersity index (PDI). FIG. 29 is a graph showing the measure particle size distribution for the T3 LNP sample, as measured by DLS.

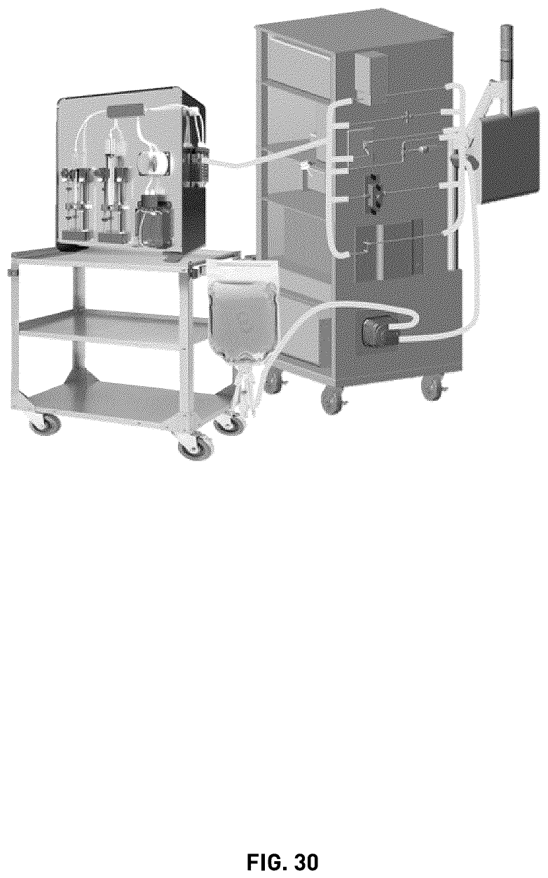

FIG. 30 illustrates a PAT Box Apparatus monitoring a bioprocess product flow stream. The apparatus is situated directly on a unit op outlet monitoring real time quality attributes as the process fluid flows into an intermediate collection container. This illustrates the set-up of MALS with the LNP mixing skid outlet process flow stream and the data demonstrated in Example 4 to measure mRNA-LNP particle size, PDI, and particle concentration.

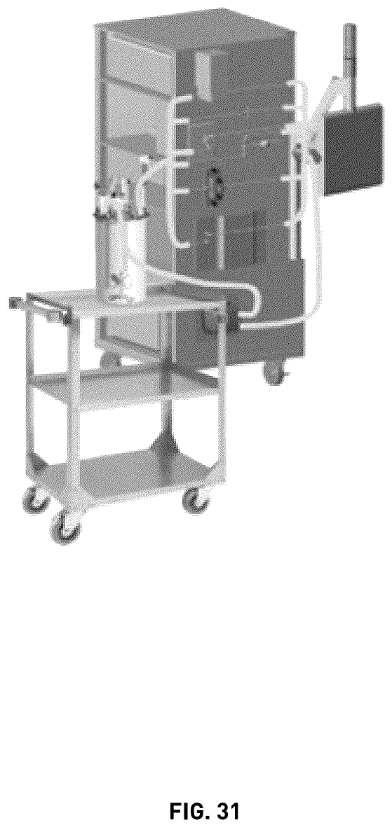

FIG. 31 illustrates a PAT Box Apparatus monitoring quality attributes and reaction kinetics of bioprocess reaction vessel containing reagents and buffer that produce active product ingredients and/or drug substances. This illustrates the set-up of Raman with the IVT reaction vessel to measure mRNA growth kinetics and NTP consumption kinetics and the data demonstrated in Example 1. This also illustrates the set-up of Mid IR with the IVT reaction vessel and the data demonstrated in Example 3 to measure mRNA concentration (mg/mL).

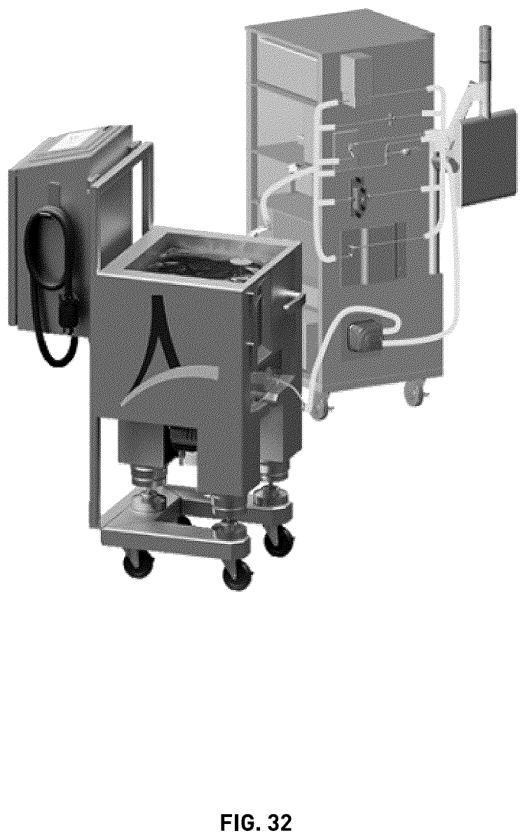

FIG. 32 illustrates PAT Box Apparatus monitoring quality attributes of a bioprocess product intermediate bulk in a surge vessel or other intermediate holding container typically positioned between unit operations. This illustrates the set-up of Raman with the IVT pool vessel, TFF1 pool vessel, and TFF 2 pool vessel and the data demonstrated in Example 1 to measure mRNA concentration (mg/mL). This also illustrates the set-up of FlowVPX (UV spectrophotometer) with the TFF1 pool vessel, OligodT Chromatography pool vessel, and TFF 2 pool vessel and the data demonstrated in Example 2 to measure mRNA concentration (mg/mL). This also illustrates the set-up of MALS with the LNP pool vessel and the data demonstrated in Example 4 to measure mRNA-LNP particle size, PDI, and particle concentration. This also illustrates the set-up of In-line DLS with the LNP pool vessel and the data demonstrated in Example 5 to measure mRNA-LNP particle size, and PDI.

In the context of the foregoing, it was necessary to solve the technical problem of arranging all of the PAT instruments as close together as possible to create a compact modular unit adapted for moving quickly and easily between unit operations of the mRNA manufacturing process. The PAT instruments were placed in a stacked configuring which allowed for their respective flow cells to be connected either in series or in parallel. This configuration also permitted the inlet to the first flow cell and the outlet to the last flow cell to be more closely situated to the recirculation tubing connections to the surge vessel or reaction vessel, thus minimizing the recirculation loop length. As discussed above, minimizing recirculation loop length reduces hold up volume which minimizes quality implications to the IVT reactor in-process bulk. Reducing hold up also reduces yield loss and thus maximizes the value of the bioprocess fluid by reducing waste and reducing cost of goods.

The configuration of the PAT instruments into individually stacked decks also allows the PAT instruments to be added or removed quickly so that only the instrument(s) necessary to measure the CQAs at a particular unit operation are present in the apparatus. Removing unnecessary PAT instruments reduces capital cost and also reduces the stack height, thus minimizing the recirculation loop length and hold up volume. Being able to quickly reconfigure the PAT stack as needed per unit operation need also reduces downtime in the lab or cleanroom suite and maximizes the use of the operation space.

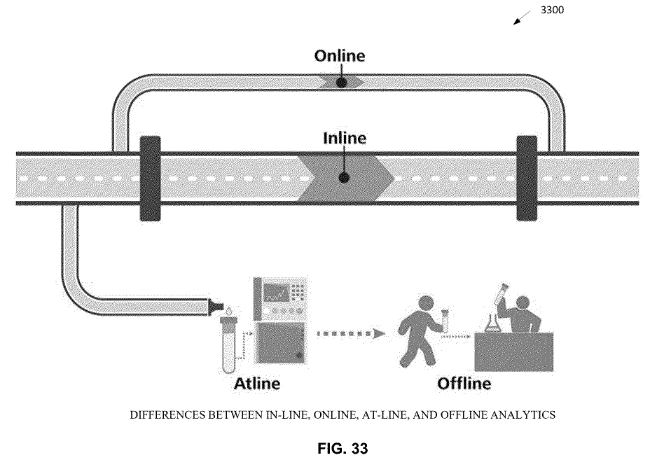

FIG. 33 illustrates an example of differences between in-line, online, at-line, and offline sample collection and analytics 3300. Analytical systems have several pathways for accepting samples of process fluid from the manufacturing process.

In-line sources of samples of the process fluid are measured as they flow through the manufacturing process. The manufacturing process is not altered in any way and continues as intended with minimal disturbance from analytical systems except for an analytical flow cell placed directly in the flow path of the manufacturing process.

Online sources of samples are taken via a side stream flow path that is diverted away from the main manufacturing process flow path and the sample runs directly into the online analytical device flow cell or sample port for data acquisition and material analysis. The remainder of the side stream process material is either directed back to the main/active manufacturing process flow path or to waste.

At-line sources of samples are taken either from the main process flow path, side stream flow path or manually via syringe and placed into a static container like a vial or conical. This static sample container is already attached to the at-line analytical device and requires little to no manual intervention to begin the analytical testing method on the at-line analytical device.

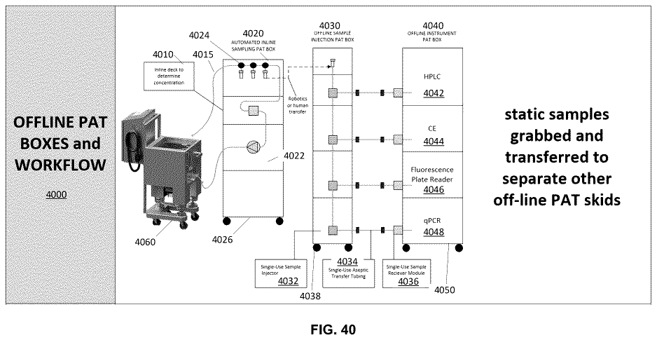

Offline sources of samples are taken either from the main process flow path, side stream flow path or manually via syringe and placed into a container like a vial or conical. Contemporary processes require the sample to be physically moved from the sampling area to another area in the same room, same facility, or another facility and manually attached to the analytical device to begin the analytical method. Embodiments herein may, advantageously, provide an automated or predominately automated process for capturing and distributing or injecting samples via a sample injector stack to an offline instrument PAT box comprising one or more process analytical instruments for offline analysis and data collection such as the offline PAT boxes and workflow shown in FIG. 40.

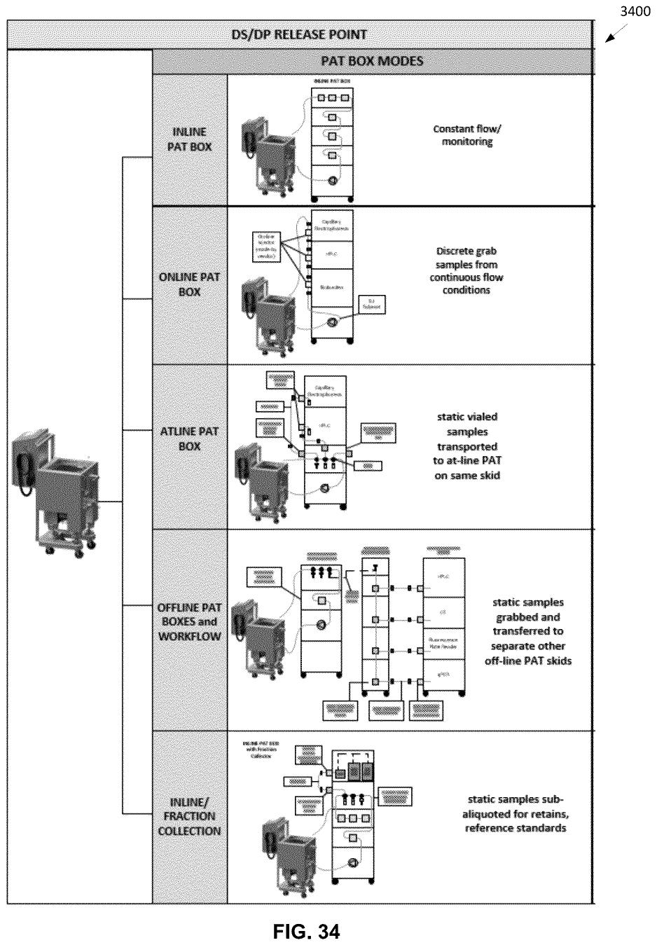

FIG. 34 illustrates various embodiments for PAT box/PAT box skid configurations 3400. The embodiments of PAT box/PAT box skid configurations include an in-line PAT box, an online PAT box, an at-line PAT box skid, and offline PAT box skid, and an in-line PAT box skid including fraction collection. The PAT box skid configurations may include a housing comprising an internal vessel in the form of a flexible or a rigid container defining an interior space suitable for holding a process fluid from the pharmaceutical or biopharmaceutical process.

Details about embodiments of PAT box and PAT box skid configurations are illustrated and discussed in more detail below. Note that these PAT box and PAT box skid configurations are examples and embodiments are not limited to these configurations.

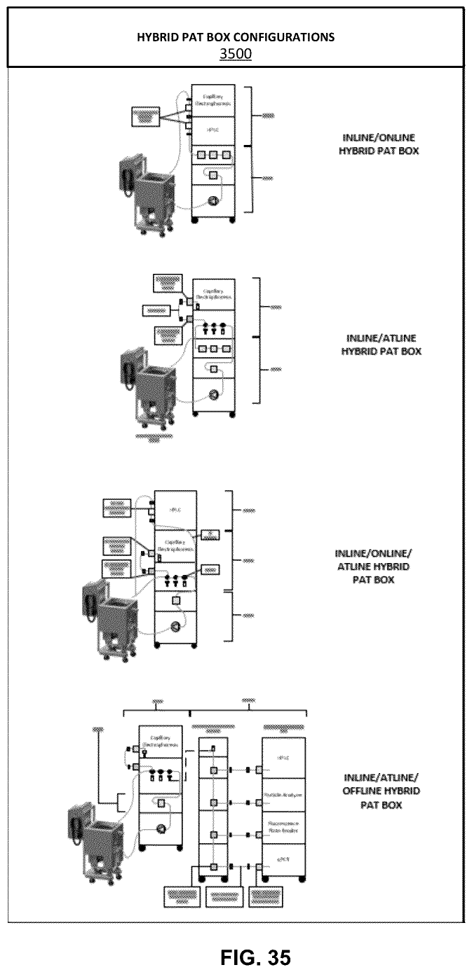

FIG. 35 illustrates various embodiments for hybrid PAT box/PAT box skid configurations 3500. The hybrid PAT box/PAT box skid configurations include an in-line/online hybrid PAT box, an in-line/at-line hybrid PAT box, an in-line/online/at-line hybrid PAT box, and an in-line/at-line/offline hybrid PAT box.

Details about embodiments of PAT box and PAT box skid configurations are illustrated and discussed in more detail below. Note that these PAT box and PAT box skid configurations are examples and embodiments are not limited to these configurations.

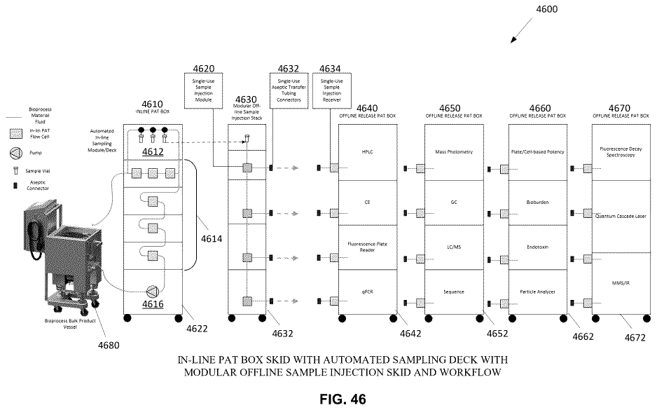

FIG. 36 illustrates two different embodiments of a PAT box skid. The top PAT box skid 3600 includes an in-line PAT box with an automated in-line sampling module, or deck. The automated in-line sampling module is connected to an offline sample injection stack PAT box with a stack of multiple single use (SU) sample injection modules. The offline sample injection stack PAT box is connected (via, e.g., SU tubsets) to multiple offline instrument PAT boxes with different offline instrument stacks. Embodiments such as this are discussed in more detail in conjunction with FIG. 46.

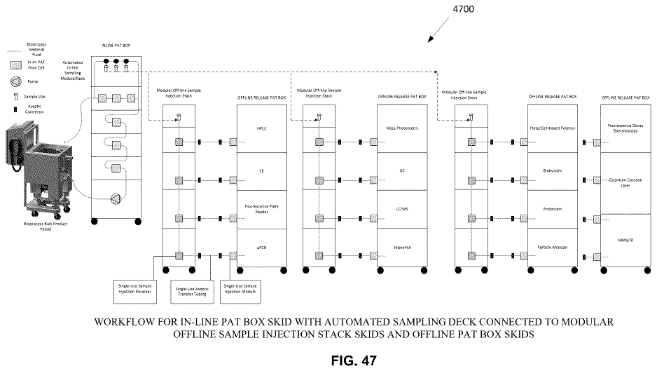

The bottom PAT box skid 3610 includes an in-line PAT box with an automated in-line sampling module, or deck. The automated in-line sampling module is connected to multiple offline sample injection stack PAT boxes with stacks of multiple SU sample injection modules. The offline sample injection stack PAT boxes may connect (via, e.g., aseptic transfer tubing) to multiple offline instrument PAT boxes with different offline instrument stacks. Embodiments such as this are discussed in more detail in conjunction with FIG. 47.

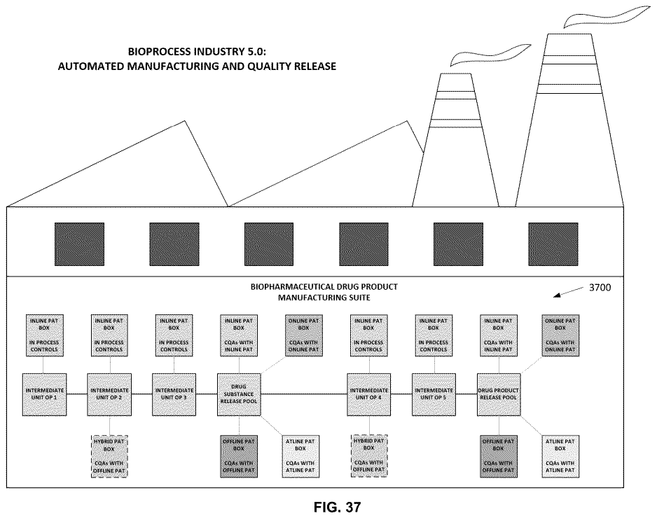

FIG. 37 illustrates an embodiment 3700 of a pharmaceutical or biopharmaceutical manufacturing process system incorporating in-line, online, at-line, and offline PAT boxes and/or PAT box skids to automate a pharmaceutical or biopharmaceutical manufacturing process. Note that the pharmaceutical or biopharmaceutical manufacturing process system shown is illustrative and embodiments of such systems are not limited to the embodiment shown.

The pharmaceutical or biopharmaceutical manufacturing process system includes multiple in-line PAT boxes to control the process flow and multiple in-line PAT boxes to measure, calculate, or estimate CQAs for the pharmaceutical or biopharmaceutical manufacturing process system at different unit operations and release pools throughout the various stages of the pharmaceutical or biopharmaceutical manufacturing process.

The pharmaceutical or biopharmaceutical manufacturing process system also includes multiple hybrid, offline, at-line, and online PAT boxes to measure, calculate, or estimate CQAs for the pharmaceutical or biopharmaceutical manufacturing process system at different unit operations or release pools throughout the various stages of the pharmaceutical or biopharmaceutical manufacturing process. Note that the placement and functions of the PAT boxes in pharmaceutical or biopharmaceutical manufacturing process system is illustrative and embodiments of layouts for such systems are not limited to the embodiment shown.

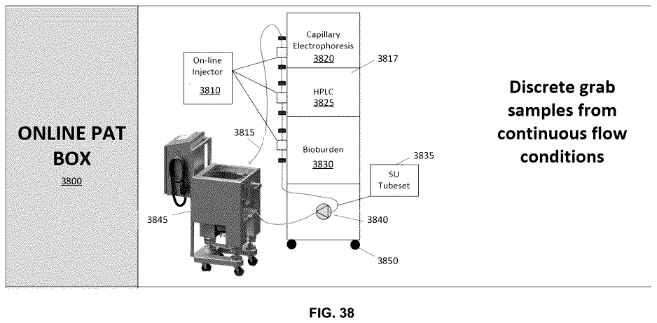

FIG. 38 illustrates an embodiment of an online PAT box 3800. The on-line PAT box encloses online process analytical technology hardware 3817 or analytical systems and allows online process analytical technology hardware or analytical systems to be stacked vertically (shown) and/or horizontally (not shown) in a re-configurable manner. A process flow path is connected to a unit operation or intermediate vessel recirculation line 3815 to an online sample injector 3810 that transfers a grab sample from the flow path to the process analytical technology hardware 3817. A purpose of the online PAT Box is to directly connect the process 3845 to process analytical technology hardware 3817 through automation and closed process lines. The online samples may flow through SU tubesets 3835 to on-line injectors, or sample ports, for each of the instruments in the on-line PAT box for data acquisition and material analysis. A pump 3840 of the online PAT box may return the samples to the process or direct the samples to waste. The pump 3840 may be part of a power, data, and pump deck to provide power to the PAT box and communicate data to and from the equipment in the PAT box in addition to pumping process fluid such as the samples to and/or from the process 3845.

The on-line PAT box may include a housing 3850 comprising process analytical technology hardware. The online process analytical technology hardware may comprise a human machine interface (not shown), a controller (not shown), and a data acquisition and analysis software layer (not shown), and an instrument stack (3820, 3825, and 3830) to detect data associated with critical quality attributes in samples from the pharmaceutical or biopharmaceutical manufacturing process. The instrument stack comprises one or more process analytical instruments coupled with the data acquisition and analysis software layer to provide the data associated with one or more critical quality attributes of the samples. In the present embodiment, the on-line instrument stack includes one or more one or more process analytical instruments such as a capillary electrophoresis (CE) instrument 3820, a high performance liquid chromatography (HPLC) instrument 3825, and a bioburden instrument 3830.

The controller may comprise processing circuitry to execute the data acquisition and analysis software to analyze the data collected from the one or more process analytical instruments, to execute one or more instrument models to model the one or more process analytical instruments; and to execute an analysis model to determine information about the one or more critical quality attributes of the samples.

The human machine interface may communicate the information about the one or more critical quality attributes (CQAs) via a local display device and/or a remote display device. For instance, the HMI may display the current CQAs, a historical trend of the values for the CQAs for a time period during the process, and/or a prediction, generated by a CQA prediction model, for subsequent measurements of the CQAs from samples during the process.

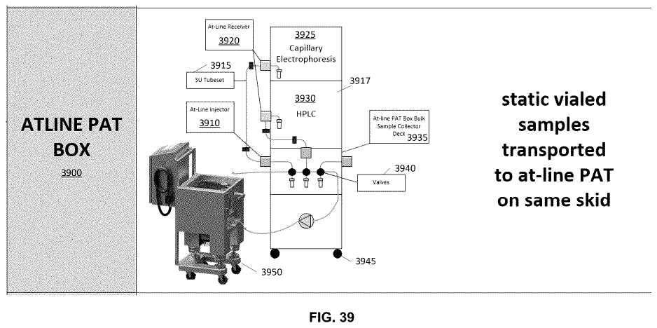

FIG. 39 illustrates an embodiment of an at-line PAT box 3900. The at-line PAT box 3900 encloses at-line process analytical technology hardware 3917 or analytical systems and allows the at-line process analytical technology hardware 3917 to be stacked vertically and/or horizontally in a re-configurable manner. A process flow path is connected to a unit operation or intermediate vessel recirculation line via an SU tubeset 3915 to an at-line sample injector 3910 that transfers a grab sample from the flow path and delivers the grab sample directly to the at-line process analytical technology hardware 3917 via a sample port or receiver module 3920 for data acquisition and material analysis. The SU tubesets, such as the SU tubeset 3915, are designed to mitigate any adverse affects to the material in the flow path. Such mitigations include, but are not limited to insulation, active heat exchangers, changes of the size of the tubing outer and inner diameters of the tubeset, and configurations to decrease the amount of inner flow path size expansions or reductions to reduce shear forces. Accommodations can also be made to the flow rate of the flow path by configurations to modulate the flow rate and pressure produced by a pump connected to the flow path. A purpose of the at-line PAT box is to directly connect the process to at-line process analytical technology hardware 3917 through automation and closed process lines.

The at-line PAT box may include a housing 3945 comprising at-line process analytical technology hardware 3917. The at-line process analytical technology hardware 3917 may comprise a human machine interface, a controller, and a data acquisition and analysis software layer, and an instrument stack to detect data associated with critical quality attributes in samples from the pharmaceutical or biopharmaceutical manufacturing process. The instrument stack (3925 and 3930) comprises one or more process analytical instruments coupled with the data acquisition and analysis software layer to provide the data associated with one or more critical quality attributes of the samples. In the present embodiment, the on-line instrument stack includes one or more one or more process analytical instruments such as a CE instrument 3925 and an HPLC instrument 3930.

In some embodiments, the at-line process analytical technology hardware 3917 may also comprise an at-line bulk sample collector 3935 and valves 3940. The sample injectors may comprise a first at-line receiver 3920 coupled with the CE 3925 (or other instrument) and the SU tubeset 3915, the at-line injector 3910 coupled between the first at-line receiver 3920 and the at-line bulk sample collector 3935 via the SU tubeset 3915, and a second at-line receiver 3920 coupled between the HPLC instrument 3930 (or other instrument) and the at-line bulk sample collector 3935.

FIG. 40 illustrates an embodiment of a workflow 4000 with offline PAT boxes. The offline PAT box encloses offline process analytical technology hardware 4040 with a power, data, and pump deck 4022 and allows the offline process analytical technology hardware 4040 to be stacked vertically and/or horizontally in a re-configurable manner. The power, data, and pump deck 4022 may provide power to the PAT boxes (or PAT box skid) and communicate data to and from the equipment in the PAT boxes in addition to pumping process fluid such as the samples to and/or from the process 4060.

A process flow path is connected to a unit operation or intermediate vessel recirculation line via a SU tubeset 4015 to a sample port such as the valves 4024 that transfers a grab sample from the flow path to the offline process analytical technology hardware 4040 via a sample port, or receiver module 4036 for data acquisition and material analysis. A purpose of the offline PAT boxes 4030 and 4040 is to directly connect the process to offline process analytical technology hardware through automation and closed process lines.

The offline PAT boxes 4030 and 4040 may include a housing 4038 and 4050 comprising offline process analytical technology hardware. The offline process analytical technology hardware may comprise a human machine interface, a controller, and a data acquisition and analysis software layer, and a sample injector stack 4030 to provide samples to instruments in an instrument stack of the offline instrument PAT box 4040 to detect data associated with critical quality attributes in samples from the pharmaceutical or biopharmaceutical manufacturing process 4060. The sample injector stack may include a stack of one or more in-line sample injector modules 4032 such as SU sample injector modules that couple with the offline instrument process analytical technology box 4040 via aseptic transfer tubing 4034 and the SU sample receiver module 4036. In some embodiments, the sample injector stack may comprise probes, sensors, or the like.

The instrument stack comprises one or more process analytical instruments coupled with the data acquisition and analysis software layer to provide the data associated with one or more critical quality attributes of the samples. In the present embodiment, the on-line instrument stack includes one or more one or more process analytical instruments such an HPLC instrument 4042, a CE instrument 4044, a fluorescence plate reader 4046, a quantitative polymerase chain reaction (qPCR) system 4048, a mass photometry instrument, a gas chromatograph (GC) instrument, a liquid chromatography-mass spectroscopy (LC-MS) instrument, a nucleic acid sequencer (sequence), a plate/cell-based potency instrument, a bioburden instrument, an endotoxin instrument, a particle analyzer, a fluorescence decay spectroscopy instrument, a quantum cascade laser instrument, a Microfluidic Modulation/Infrared Spectroscopy (MMS/IR) instrument, or a combination thereof.

FIG. 41 illustrates an in-line PAT box with a fraction collector 4145 (aka a fraction collector PAT box 4100). The fraction collector PAT box 4100 may enclose in-line process analytical technology hardware 4160 and the fraction collector 4145 that allows the in-line process analytical technology hardware 4160 and a fraction collector 4145 to be stacked vertically and/or horizontally in a re-configurable manner. The fraction collector PAT box 4100 may also include a power, data, pump deck 4125. The pump 4125 may be part of a power, data, and pump deck to provide power to the PAT box and communicate data to and from the equipment in the PAT box in addition to pumping process fluid such as the samples to and/or from the process 4170.

A process flow path is connected via an SU tubeset 4115 to a unit operation or intermediate vessel recirculation line to the sample ports such as the at-line injector 4110 and a fraction collector receiver 4120 to measure attributes such as analyte concentration in real time and in-line process analytical technology hardware 4160 that transfers a grab sample from the flow path to the fraction collector 4145 via the sample receiver module 4120. A purpose of the fraction collector PAT box 4100 is to collect representative process samples for retain or reference standards. In some embodiments, the fraction collector 4145 comprises an inline sample collector deck with a bulk collector 4130, a well plate instrument 4135, and a vial plate instrument 4140.

The fraction collector PAT box 4100 may also include a housing 4165 comprising in-line process analytical technology hardware 4160 and a fraction collector 4145. The in-line process analytical technology hardware 4160 may comprise a human machine interface, a controller, and a data acquisition and analysis software layer, and an instrument stack to detect data associated with critical quality attributes in samples from the pharmaceutical or biopharmaceutical manufacturing process 4170. The instrument stack comprises one or more process analytical instruments coupled with the data acquisition and analysis software layer to provide the data associated with one or more critical quality attributes of the samples. In the present embodiment, the on-line instrument stack includes one or more one or more process analytical instruments such as the well plate instrument 4135 and vial plate instrument 4140.

There are also many possible configurations of Hybrid PAT Boxes which can enclose certain combinations of in-line, at-line, offline process analytical technology hardware or analytical systems and a fraction collector that allows them to be stacked vertically in a re-configurable manner. A process flow path is connected to a unit operation or intermediate vessel recirculation line to the inline flow cell to measure attributes such as analyte concentration in real time and an online/at-line/offline line sample injector that transfers a grab sample from the flow path to the online/at-line/offline analytical system or fraction collector system via a sample receiver module. A purpose of the Hybrid PAT boxes is to give flexibility to use any type of process analytical technology hardware or analytical system for virtually any specific application regardless of whether it is in-line, online, at-line, or offline.

The online, at-line, offline, fraction collector, and hybrid PAT boxes along with the in-line PAT box completes a family of PAT boxes that brings the dream closer to reality of bringing all analytical systems needed for in-process control and release testing criteria out of the lab and directly connected to manufacturing via hardware and software. This technology enables the future of smarter biopharmaceutical manufacturing facilities, reduces operating expenses, and drives higher throughput of products through the facility by using higher degrees of automation. The on-line, at-line, offline, fraction collector, and hybrid PAT boxes along with the in-line PAT box brings current analytical development and quality control out of a manual and slow dynamic into a smarter more efficient thus producing a “Smart Manufacturing” workflow such as the workflow shown in FIG. 37.

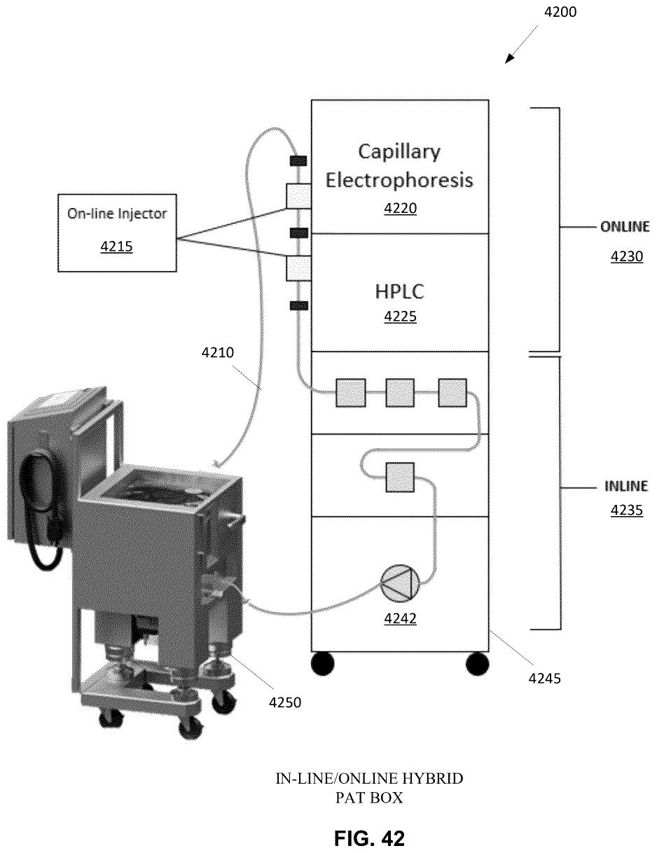

FIG. 42 illustrates a hybrid in-line/online hybrid PAT box 4200 comprising hybrid online and in-line process analytical technology hardware. The hybrid online and in-line process analytical technology hardware comprises online process analytical instruments such as a CE instrument 4220 and an HPLC instrument 4225. A SU tubeset 4210 connects a first on-line sample injector 4215 with a second on-line sample injector 4215. In many embodiments, the PAT box includes a power, data, and pump deck 4242 to provide power to the PAT box and communicate data to and from the equipment in the PAT box in addition to pumping process fluid such as the samples to and/or from the process 4250.

In the present embodiment, the first on-line sample injector 4215 is coupled with CE instrument 4220 (or other instrument) and the second on-line injector 4215 is coupled with the HPLC instrument 4225 (or other instrument). The hybrid online and in-line process analytical technology hardware further comprises in-line process analytical instruments 4235 including one or more spectroscopic instruments selected from a Raman spectrophotometer, a mid-IR spectrophotometer, a near-IR spectrophotometer, and an V-VIS spectrophotometer. The in-line process analytical instruments 4235 couple with the SU tubeset 4210. In some embodiments, the inline process analytical instruments 4235 further comprises a multiangle light scattering (MALS) detector coupled with the SU tubeset.

The hybrid in-line/online hybrid PAT box 4200 may also include a housing 4245 comprising hybrid online and in-line process analytical technology hardware. The hybrid online and in-line process analytical technology hardware may comprise a human machine interface, a controller, and a data acquisition and analysis software layer, and an instrument stack to detect data associated with critical quality attributes in samples from the pharmaceutical or biopharmaceutical manufacturing process 4250. The instrument stack comprises one or more process analytical instruments coupled with the data acquisition and analysis software layer to provide the data associated with one or more critical quality attributes of the samples of process fluid. In the present embodiment, the instrument stack comprises the on-line process analytical instruments 4230 and the in-line process analytical instruments 4235.

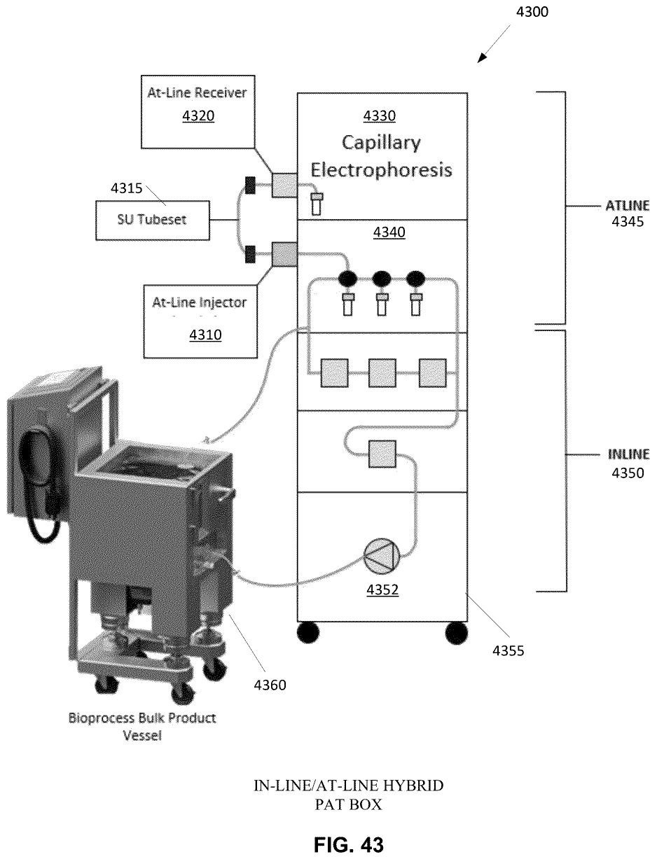

FIG. 43 illustrates an embodiment of a hybrid in-line/at-line PAT box 4300. The hybrid in-line/at-line PAT box 4300 may comprise hybrid at-line and in-line process analytical technology hardware. The hybrid at-line and in-line process analytical technology hardware comprises an at-line process analytical instrument such as a CE instrument 4330 and an at-line bulk collector sample deck 4340. A tubeset 4315 may connect with an at-line receiver 4320 at the at-line process analytical instrument and an at-line injector 4310 at the at-line bulk collector sample deck 4340. In many embodiments, the PAT box includes a power, data, and pump deck 4352 to provide power to the PAT box and communicate data to and from the equipment in the PAT box in addition to pumping process fluid such as the samples to and/or from the process 4360.

The hybrid in-line/at-line PAT box 4300 may also include a housing 4355 comprising hybrid at-line and in-line process analytical technology hardware. The hybrid at-line and in-line process analytical technology hardware may comprise a human machine interface, a controller, and a data acquisition and analysis software layer, and an instrument stack to detect data associated with critical quality attributes in samples from the pharmaceutical or biopharmaceutical manufacturing process 4360. The instrument stack comprises one or more process analytical instruments coupled with the data acquisition and analysis software layer to provide the data associated with one or more critical quality attributes of the samples. In the present embodiment, the instrument stack comprises an at-line process analytical instrument stack 4335 and an inline process analytical instrument stack 4350.

The in-line process analytical instrument stack 3350 may include one or more spectroscopic instruments selected from a Raman spectrophotometer, a mid-IR spectrophotometer, a near-IR spectrophotometer, and an UV-VIS spectrophotometer.

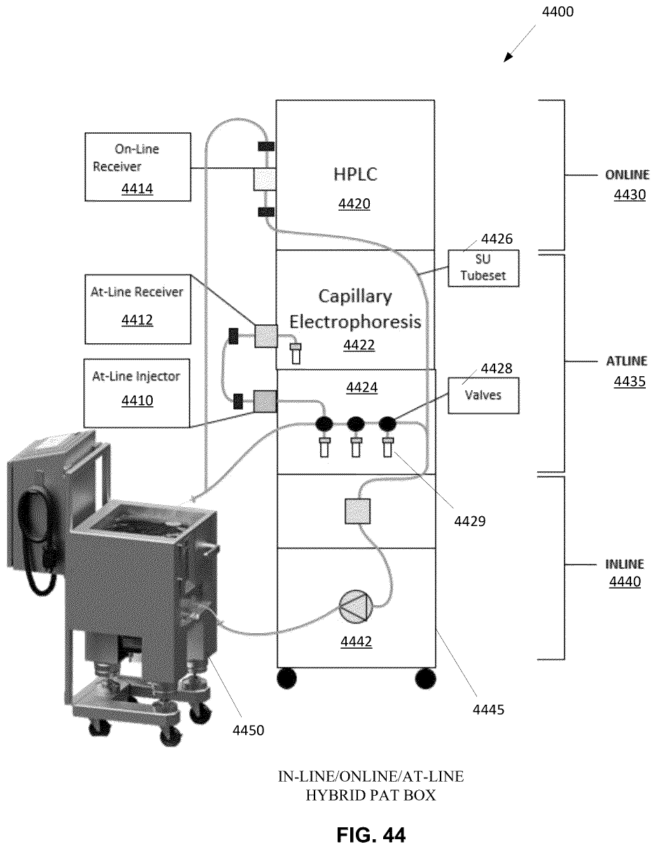

FIG. 44 illustrates an embodiment of a hybrid in-line/online/at-line PAT box 4400. The hybrid in-line/online/at-line PAT box 4400 may comprise hybrid online, at-line, and in-line process analytical technology hardware. The hybrid online, at-line, and in-line process analytical technology hardware comprises an online HPLC instrument 4420, an at-line CE instrument 4422, and an at-line bulk collector sample deck 4424. A tubeset 4426 may connect with an online receiver 4414 at the HPLC instrument 4420, an at-line receiver 4412 at the CE instrument 4422, and an at-line injector 4410 at the at-line bulk sample collector 4424. The at-line bulk sample collector 4424 may include valves 4428 coupled with vessels 4429 such as vials.

The hybrid in-line/online/at-line PAT box 4400 may also include a housing 4445 comprising hybrid online, at-line, and in-line process analytical technology hardware. The hybrid online, at-line, and in-line process analytical technology hardware may comprise a human machine interface, a controller, and a data acquisition and analysis software layer, and an instrument stack to detect data associated with critical quality attributes in samples from the pharmaceutical or biopharmaceutical manufacturing process 4450. The instrument stack comprises one or more process analytical instruments coupled with the data acquisition and analysis software layer to provide the data associated with one or more critical quality attributes of the samples. In the present embodiment, the instrument stack includes an online process analytical instrument stack 4430, an at-line process analytical instrument stack 4435, and an in-line process analytical instrument stack 4440. In many embodiments, the PAT box includes a power, data, and pump deck 4442 to provide power to the PAT box and communicate data to and from the equipment in the PAT box in addition to pumping process fluid such as the samples to and/or from the process 4450.

The in-line process analytical instrument stack 4440 may include one or more spectroscopic instruments selected from a Raman spectrophotometer, a mid-IR spectrophotometer, a near-IR spectrophotometer, and an UV-VIS spectrophotometer.

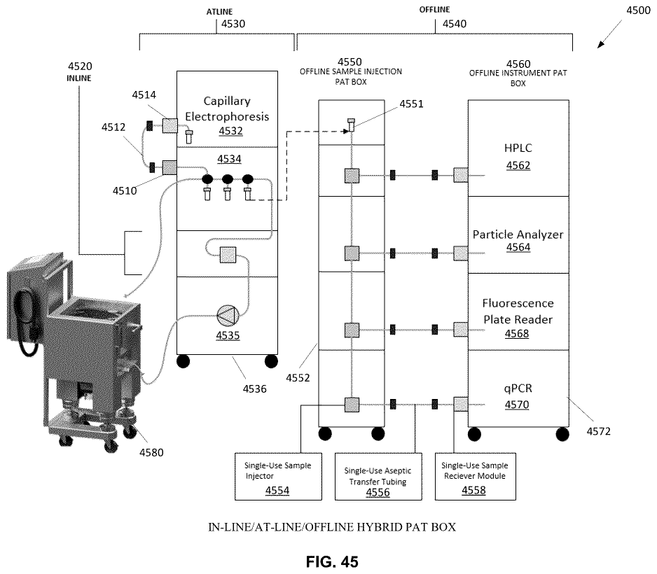

FIG. 45 illustrates a hybrid in-line/offline/at-line PAT box 4500. The hybrid in-line/offline/at-line PAT box encloses hybrid online, at-line, and offline process analytical technology hardware. The hybrid online, at-line, and offline process analytical technology hardware may comprise an at-line CE instrument 4532, an at-line bulk collector sample deck 4534, one or more in-line process analytical instruments 4520, and one or more offline sample injection process analytical technology boxes 4550 coupled with one or more offline instrument process analytical technology boxes 4560. In many embodiments, the PAT box includes a power, data, and pump deck 4535 to provide power to the PAT box and/PAT box skid and communicate data to and from the equipment in the PAT box and/PAT box skid in addition to pumping process fluid such as the samples to and/or from the process 4580.

The hybrid in-line/offline/at-line PAT box 4500 may also include a housing 4536, 4552, and 4572 comprising hybrid in-line, offline, and at-line process analytical technology hardware. The hybrid online, offline, and at-line process analytical technology hardware may comprise a human machine interface, a controller, and a data acquisition and analysis software layer, and an instrument stack to detect data associated with critical quality attributes in samples from the pharmaceutical or biopharmaceutical manufacturing process 4580. The instrument stack comprises one or more process analytical instruments coupled with the data acquisition and analysis software layer to provide the data associated with one or more critical quality attributes of the samples. In the present embodiment, the instrument stack includes the at-line instrument stack 4530, the in-line instrument stack 4520, the offline instrument stack in the offline instrument PAT box 4560.

The offline instrument PAT box 4560 may include offline process analytical instruments such as an HPLC instrument 4562, a particle analyzer instrument 4564, a fluorescence plate reader 4568, a qPCR system 4570, a mass photometry instrument, a GC instrument, a LC-MS instrument, a nucleic acid sequencer (sequence), a plate/cell-based potency instrument, a bioburden instrument, an endotoxin instrument, a fluorescence decay spectroscopy instrument, a quantum cascade laser instrument, and a MMS/IR instrument.

The hybrid online, at-line, and in-line process analytical technology hardware may further comprise in-line process analytical instruments 4520 including one or more spectroscopic instruments selected from a Raman spectrophotometer, a mid-IR spectrophotometer, a near-IR spectrophotometer, and an UV-VIS spectrophotometer.

The offline sample injection PAT box 4550 may receive the samples of the process fluid at a vessel 4551 from the at-line bulk collector sample deck 4534 via various methods such as a robotic mechanism, a manual transfer, tubing, or the like. SU sample injectors may transfer the samples to offline process analytic instruments in the offline instrument PAT box 4560 through SU aseptic transfer tubing 4556 via SU receiver modules 4558 at the offline process analytic instruments such as the HPLC 4562, the particle analyzer 4564, the fluorescence plate reader 4568, and the qPCR 4570.

FIG. 46 illustrates an embodiment of a workflow 4600 for an in-line PAT box skid with automated sampling deck and a modular offline sample injection skid discussed in conjunction with FIG. 36. The in-line PAT box skid with automated sampling deck and a modular offline sample injection skid includes an in-line PAT box 4610 with an automated in-line sampling module 4612, connected to an offline sample injection stack PAT box 4630 with a stack of multiple SU sample injection modules 4620. The offline sample injection stack PAT box 4630 is connected (via, e.g., SU aseptic transfer tubing) to multiple offline instrument PAT boxes 4640, 4650, 4660, and 4670 with different offline instrument stacks. Each of the multiple SU sample injection modules 4620 of the offline sample injection stack PAT box 4630 is connected (via, e.g., SU aseptic transfer tubing) to a different offline instrument of the offline instrument stacks. In some embodiments, the offline sample injection stack PAT box 4630 includes an HMI comprising a controller and a software layer to control the timing, volume, etc., of the samples provided to the different offline instruments of the offline instrument PAT boxes 4640, 4650, 4660, and 4670. In many embodiments, the PAT box includes a power, data, and pump deck 4616 to provide power to the PAT box and/or PAT box skid and communicate data to and from the equipment in the PAT box and/or PAT box skid in addition to pumping process fluid such as the samples to and/or from the process 4680.

The in-line PAT box skid with automated sampling deck, offline sample injection PAT box 4630, and one or more of the different offline instrument PAT boxes 4640, 4650, 4660, and 4670 may each include a housing 4622, 4632, 4642, 4652, 4662, and 4672 comprising process analytical technology hardware. The process analytical technology hardware may comprise a human machine interface, a controller, and a data acquisition and analysis software layer, and an instrument stack or a sample injection module stack to process samples from the pharmaceutical or biopharmaceutical manufacturing process 4680. In other embodiments, the in-line PAT box 4610, offline sample injection module PAT box 4630, and offline instrument PAT boxes 4640, 4650, 4660, and 4670 may comprise part of a skid that includes a single human machine interface, a controller, and a data acquisition and analysis software layer to manage the in-line PAT box 4610, offline sample injection module PAT box 4630, and offline instrument PAT boxes 4640, 4650, 4660, and 4670 that are part of the skid.

The instrument stack comprises one or more process analytical instruments coupled with the data acquisition and analysis software layer for data acquisition and material analysis to provide the data associated with one or more critical quality attributes of the samples. In the present embodiment, the inline instrument stack may include one or more one or more process analytical instruments such as a Raman spectrophotometer, a mid-IR spectrophotometer, a near-IR spectrophotometer, and an UV-VIS spectrophotometer. The offline process analytical instruments may include instruments such as a HPLC instrument, a CE instrument, a fluorescence plate reader, a qPCR system, a mass photometry instrument, a GC instrument, a LC-MS instrument, a nucleic acid sequencer (sequence), a plate/cell-based potency instrument, a bioburden instrument, an endotoxin instrument, a particle analyzer, a fluorescence decay spectroscopy instrument, a quantum cascode laser instrument, and a MMS/IR instrument.

FIG. 47 illustrates an embodiment of a workflow 4700 with an in-line PAT box with an automated in-line sampling module, or deck, which is discussed in conjunction with FIG. 36. The automated in-line sampling module is connected to multiple offline sample injection stack PAT boxes with stacks of multiple SU sample injection modules. The offline sample injection stack PAT boxes may connect (via, e.g., SU tubsets) to multiple offline instrument PAT boxes with different offline instrument stacks. Each of the multiple SU sample injection modules of the offline sample injection stack PAT box is connected (via, e.g., SU aseptic transfer tubing) to a different offline instrument of the offline instrument stacks of the multiple offline instrument PAT boxes. In some embodiments, the offline sample injection stack PAT boxes include controllers and software layers to control the timing, volume, etc., of the samples provided to the different offline instruments of the offline instrument PAT boxes.

Similar to the inline and offline PAT boxes described in conjunction with FIG. 46, the in-line PAT box skid with automated sampling deck and one or more of the different offline instrument PAT boxes may each include a housing comprising process analytical technology hardware. The process analytical technology hardware may comprise a human machine interface, a controller, and a data acquisition and analysis software layer, and an instrument stack to detect data associated with critical quality attributes in samples from the pharmaceutical or biopharmaceutical manufacturing process. In other embodiments, the in-line PAT box and offline instrument PAT boxes may comprise part of a skid that includes a single human machine interface, a controller, and a data acquisition and analysis software layer to manage the in-line PAT box and offline instrument PAT boxes.

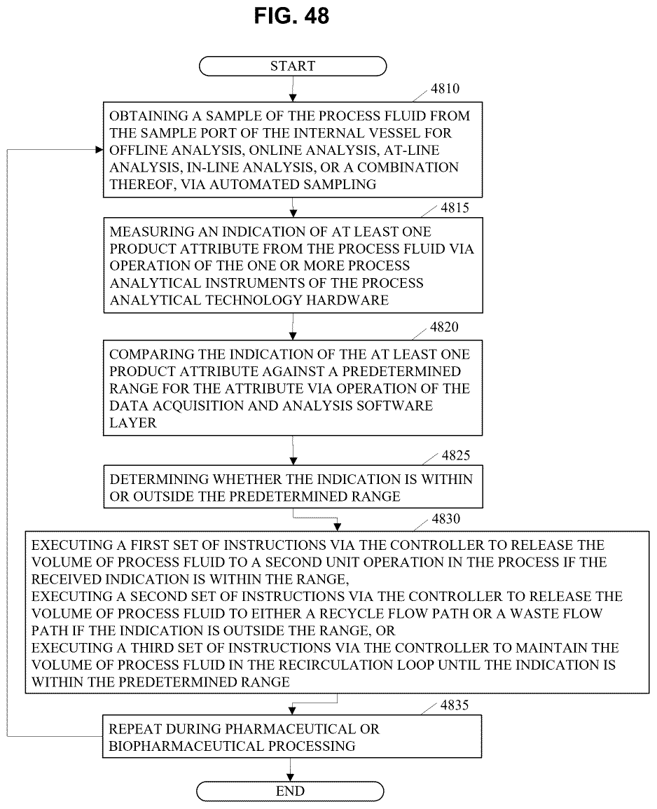

FIG. 48 illustrates an embodiment of a method as a flowchart in accordance with one embodiment. The method is for real time release testing in a pharmaceutical or biopharmaceutical process. The method may comprise connecting one or more PAT boxes or PAT box skids to a unit operation of the pharmaceutical or biopharmaceutical process to capture process fluid for samples.

The method includes obtaining or capturing a sample of the process fluid from the sample port of the internal vessel for offline data acquisition and material analysis, online data acquisition and material analysis, at-line data acquisition and material analysis, in-line data acquisition and material analysis, or a combination thereof, via automated sampling (element 4810). For instance, a Modular Automated Sampling Technology (MAST) device, such as the MAST illustrated in FIG. 16, may capture samples from a pharmaceutical or biopharmaceutical process for at-line, online, and offline PAT boxes. A flow cell may direct a flow of a recirculation process for capturing samples for in-line PAT boxes with a recirculation loop of the unit operation. In embodiments, the MAST device may deposit the samples in a vessel such as the vial containers 4612 shown in FIG. 46 and the samples may be captured or obtained from the bioprocess bulk product vessel 4680.

After capturing the samples, the process analytical technology hardware of a PAT box may measure an indication of at least one product attribute from the process fluid via operation of the one or more process analytical instruments (element 4815). For instance, the one or more process analytical instruments may include in-line process analytical instruments, at-line process analytical instruments, offline process analytical instruments, or online process analytical instruments.

After measuring an indication of at least one critical quality attribute, the process analytical technology hardware of a PAT box may compare the indication of the at least one product attribute against a predetermined range for the attribute via operation of the data acquisition and analysis software layer (element 4820). For instance, the predetermined range may relate to process control to allow the process fluid to progress to a subsequent unit operation in the pharmaceutical or biopharmaceutical process. The predetermined range may relate to values representing a concentration, a particle size, or a purity of the process fluid, or the like.

After measuring an indication of at least one product attribute such as a critical quality attribute, a controller of the PAT hardware may determine whether the indication is within or outside the predetermined range (element 4825). The controller may determine a set of instructions to execute based on the whether the indication is within or outside the predetermined range. The controller may execute a first set of instructions to release the volume of process fluid to a subsequent unit operation in the pharmaceutical or biopharmaceutical manufacturing process if the received indication is within the range. The controller may execute a second set of instructions to release the volume of process fluid to either a recycle flow path or a waste flow path if the indication is outside the range. Or the controller may execute a third set of instructions to maintain the volume of process fluid in the recirculation loop until the indication is within the predetermined range (element 4830).

The method may repeat by collecting another sample at element 4810. Once the indication is within the predetermined range and the process fluid is released to a subsequent unit operation in the pharmaceutical or biopharmaceutical manufacturing process, the method may repeat at element 4810 with a new batch of process fluid in the unit operation.

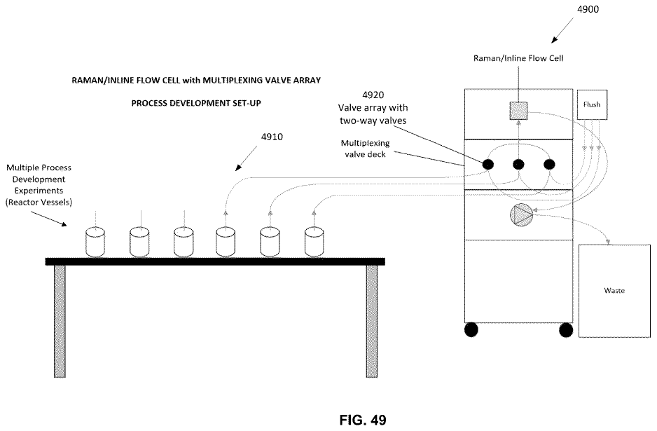

FIG. 49 illustrates an embodiment of the PAT box skid 4900 that allows for multiple reaction vessels 4910 to be sampled and directed to an inline, at-line, on-line, offline, or fraction collector PAT box or PAT box skid for measurement of quality attributes. In process development, experiments of multiple configurations of process conditions may be screened to find an optimal condition for quality and yield. In some circumstances, the number of configurations that may be screened are large enough that it necessitates multiple experiments to be completed or run simultaneously. During these runs, the PAT box skid 4900 may measure quality attributes at the beginning, during, and end of the run. In conventional systems, these quality attributes are measured by offline analytics, which can be manual, slow, and unautomated. The PAT box skid 4900 advantageously presents a small footprint and faster automated solution. An automated valve array 4920 may send samples of experiments running simultaneously from reaction vessels 4910, for example, to one inline flow cell on the PAT box skid 4900. FIG. 49 presents one such arrangement where multiple reaction vessels 4910 are connected to a valve array 4920 that allows a sample to be pulled from each vessel one at a time by the PAT box skid 4900. First, one vessel may be sampled and run the sample through the inline flow cell. Then, the sample lines can be flushed with a flush or cleaning solution. Then, the next vessel may be quickly sampled after the flush is complete and send the material to the inline flow to repeat the action. Thus, multiple reaction vessels 4910 or experiments can be sampled and measured with an inline flow in an automated fashion. The valve array 4920 can be designed as an additional deck that is a part of the PAT skid 4900. Just as there are discrete decks housing disparate analytical technologies where those decks can be added or removed as necessary, the valve array 4920 deck can be added or removed as necessary. In this process development example, the valve array 4920 deck could be added to facilitate sample multiplexing. A process development setting is one example; however, the sample multiplexing application can be used in other environments and applications as required.

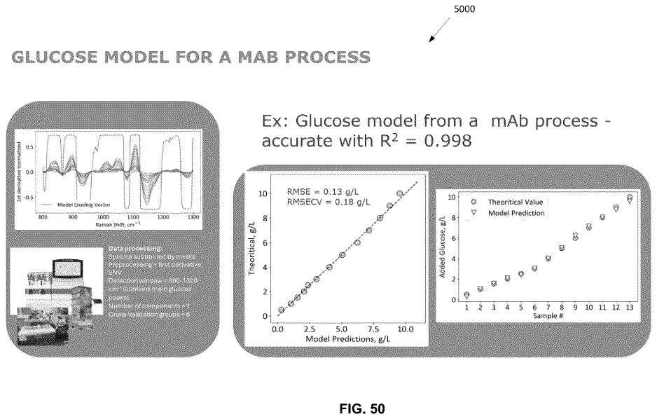

FIG. 50 shows multiple figures that outline a Raman spectrophotometer model 5000 for measuring glucose in monoclonal antibody (mAb) manufacturing process. Raman spectrophotometry can be used to measure multiple different analytes within a matrix of compounds in a manufacturing process. During a mAb manufacturing process, glucose is the main carbon source for cell growth. Therefore, glucose levels are critical quality attributes (CQAs) to be kept at optimum levels, or within optimal level ranges, during the cell culture unit operation. Conventionally, glucose is measured using offline analytics where samples must be pulled and transported to the offline analytical system. Utilizing a Raman probe either in the cell culture vessel, inline with a flow cell in a recirculated sample path, or with an adapted flow cell that prevents fouling, a PAT box can use a glucose model in conjunction with the Raman probe to determine glucose levels in real time. FIG. 50 summarizes the work completed to develop a model to measure glucose levels. The left-hand figure shows the Raman spectrum with a normalized first derivative. The middle figure shows a linear fit comparing theoretical values against the model predictions of glucose concentration in grams/liter. The right-hand figure shows the overlay of the theoretical and model predicted values of glucose concentration over 13 samples. The linear fit shows the model predicted values are accurate with an R-squared value of 0.998.

While the invention herein disclosed has been described by means of specific embodiments and applications thereof, numerous modifications and variations could be made thereto by those skilled in the art without departing from the scope of the invention set forth in the claims.

It will be appreciated that the present invention is set forth in various levels of detail in this application. In certain instances, details that are not necessary for one of ordinary skill in the art to understand the invention, or that render other details difficult to perceive may have been omitted. The terminology used herein is for the purpose of describing particular embodiments only and is not intended to be limiting beyond the scope of the appended claims. Unless defined otherwise, technical terms used herein are to be understood as commonly understood by one of ordinary skill in the art to which the disclosure belongs.

Various features of a process system may be used independently of, or in combination, with each other. It will be appreciated that a system as disclosed herein may be embodied in different forms and should not be construed as limited to the illustrated embodiments of the figures.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

In view of the above, it should be understood that the various embodiments illustrated in the figures have several separate and independent features, which each, at least alone, has unique benefits which are desirable for, yet not critical to, the presently disclosed vessel, system, and associated method. Therefore, the various separate features described herein need not all be present in order to achieve at least some of the desired characteristics and/or benefits described herein.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another.

In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An apparatus for deploying process analytical technology in a pharmaceutical or biopharmaceutical manufacturing process, the apparatus comprising:
process analytical technology hardware in a housing, wherein the process analytical technology hardware comprises a human machine interface, a controller, and a data acquisition and analysis software layer, and an instrument stack to detect data associated with critical quality attributes in samples of process fluid from the pharmaceutical or biopharmaceutical manufacturing process, wherein the instrument stack comprises one or more process analytical instruments coupled with the data acquisition and analysis software layer provide the data associated with one or more critical quality attributes of the samples; and
a sample injector to transfer the samples from the unit operation or a recirculation line to the process analytical technology hardware for analysis;
wherein the controller comprises processing circuitry to execute the data acquisition and analysis software layer to analyze the data collected from the one or more process analytical instruments, to execute one or more instrument models to model the one or more process analytical instruments; and to execute an analysis model to determine information about the one or more critical quality attributes of the samples,
wherein the human machine interface communicates the information about the one or more critical quality attributes via a display device;
wherein the process analytical technology hardware comprises online process analytical technology hardware, at-line process analytical technology hardware, offline process analytical technology hardware, a fraction collector combined with in-line process analytical technology hardware, or a hybrid thereof.

2. The apparatus of claim 1, wherein the stack further comprises an inline process analytical technology hardware and a sample injector stack within the housing or in one or more separate housings.

3. The apparatus of claim 1, wherein the process analytical technology hardware comprises the online process analytical technology hardware, wherein the online process analytical technology hardware comprises a capillary electrophoresis (CE) instrument, a high performance liquid chromatography (HPLC) instrument, a bioburden instrument, and a single use (SU) tubeset.

4. The apparatus of claim 1, wherein the process analytical technology hardware comprises the at-line process analytical technology hardware, wherein the at-line process analytical technology hardware comprises a capillary electrophoresis (CE) instrument, a high performance liquid chromatography (HPLC) instrument, an at-line bulk sample collector, valves, and a single use (SU) tubeset, wherein the sample injector comprises a first at-line receiver coupled with the CE and the SU tubeset, an at-line injector coupled between the first at-line receiver and the at-line bulk sample collector via the SU tubeset, and a second at-line receiver coupled between the HPLC and the at-line bulk sample collector.

5. The apparatus of claim 1, wherein the process analytical technology hardware comprises the offline process analytical technology hardware, wherein the offline process analytical technology hardware comprises an offline sample injection process analytical technology box with one or more single use (SU) sample injectors, coupled with an offline instrument process analytical technology box via aseptic transfer tubing.

6. The apparatus of claim 5, wherein the one or more single use (SU) sample injectors couple with two or more process analytical instruments of the offline instrument process analytical technology box comprising one or more instruments selected from a high performance liquid chromatography (HPLC) instrument, a capillary electrophoresis (CE) instrument, a fluorescence plate reader, a quantitative polymerase chain reaction (qPCR) system, a mass photometry instrument, a gas chromatograph (GC) instrument, a liquid chromatography-mass spectroscopy (LC-MS) instrument, a nucleic acid sequencer (sequence), a plate/cell-based potency instrument, a bioburden instrument, an endotoxin instrument, a particle analyzer, a fluorescence decay spectroscopy instrument, a quantum cascade laser instrument, and a Microfluidic Modulation/Infrared Spectroscopy (MMS/IR) instrument.

7. The apparatus of claim 1, wherein the process analytical technology hardware comprises the fraction collector combined with in-line process analytical technology hardware, wherein the fraction collector combined with in-line process analytical technology hardware comprises a fraction collector receiver coupled between the sample injector and a fraction collector via single use (SU) tubeset.

8. The apparatus of claim 7, wherein the fraction collector comprises a bulk collector, a well plate instrument, and a vial plate instrument, wherein the fraction collector combined with in-line process analytical technology hardware further comprises an inline sample collector deck.

9. The apparatus of claim 1, wherein the process analytical technology hardware comprises hybrid online and in-line process analytical technology hardware, wherein the hybrid online and in-line process analytical technology hardware comprises online process analytical instruments including a capillary electrophoresis (CE) instrument and a high performance liquid chromatography (HPLC) instrument, wherein single use (SU) tubeset connects a first on-line sample injector with a second on-line sample injector, the first on-line sample injector coupled with CE instrument and the second on-line injector coupled with the HPLC instrument.

10. The apparatus of claim 9, wherein the hybrid online and in-line process analytical technology hardware further comprises in-line process analytical instruments including one or more spectroscopic instruments selected from a Raman spectrophotometer, a mid-infrared (IR) spectrophotometer, a near-IR spectrophotometer, and an ultraviolet-visible (UV-VIS) spectrophotometer, wherein the in-line process analytical instruments couple with the single use (SU) tubeset.

11. The apparatus of claim 10, wherein the inline process analytical instruments further comprise a multiangle light scattering (MALS) detector.

12. The apparatus of claim 1, wherein the process analytical technology hardware comprises hybrid at-line and in-line process analytical technology hardware, wherein the hybrid at-line and in-line process analytical technology hardware comprises an at-line process analytical instrument including a capillary electrophoresis (CE) instrument and an at-line bulk collector sample deck.

13. The apparatus of claim 12, wherein the hybrid at-line and in-line process analytical technology hardware further comprises in-line process analytical instruments including one or more spectroscopic instruments selected from a Raman spectrophotometer, a mid-infrared (IR) spectrophotometer, a near-IR spectrophotometer, and an ultraviolet-visible (UV-VIS) spectrophotometer.

14. The apparatus of claim 1, wherein the process analytical technology hardware comprises hybrid online, at-line, and in-line process analytical technology hardware, wherein the hybrid online, at-line, and in-line process analytical technology hardware comprises an online high performance liquid chromatography (HPLC) instrument, an at-line capillary electrophoresis (CE) instrument, and an at-line bulk collector sample deck.

15. The apparatus of claim 12, wherein the hybrid online, at-line, and in-line process analytical technology hardware further comprises in-line process analytical instruments including one or more spectroscopic instruments selected from a Raman spectrophotometer, a mid-infrared (IR) spectrophotometer, a near-IR spectrophotometer, and an ultraviolet-visible (UV-VIS) spectrophotometer.

16. The apparatus of claim 1, wherein the process analytical technology hardware comprises hybrid online, at-line, and offline process analytical technology hardware, wherein the hybrid online, at-line, and offline process analytical technology hardware comprises an at-line capillary electrophoresis (CE) instrument and an at-line bulk collector sample deck, one or more in-line process analytical instruments, and one or more offline sample injection process analytical technology boxes coupled with one or more offline instrument process analytical technology boxes.

17. The apparatus of claim 1, wherein the process analytical technology hardware comprises in-line and offline process analytical technology hardware, wherein the hybrid in-line and offline process analytical technology hardware comprises one or more in-line process analytical instruments and one or more offline sample injection process analytical technology boxes coupled with one or more offline instrument process analytical technology boxes.

18. The apparatus of claim 1, wherein the housing comprises an internal vessel in the form of a flexible or a rigid container defining an interior space suitable for holding a fluid.

19. The apparatus of claim 1, wherein the apparatus comprises one or more environmental protections selected from electromagnetic frequency (EMF) shielding, vibrational dampening, shock absorption, and temperature control, optionally in the form of insulation.

20. The apparatus of claim 1, wherein a single use tubeset couples with the sample injector to mitigate any adverse affects to the process fluid in a flow path, wherein the single use tubset may include insulation, active heat exchangers, a changes in a size of tubing outer and inner diameters of the single use tubeset, configurations to decrease an amount of inner flow path size expansions or reductions to reduce shear forces and configurations to modify a flow rate of a flow path by modulating the flow rate and a pressure produced by a pump connected to the flow path.

21. The apparatus of claim 1, wherein the sample port is adapted to be connected with the recirculation loop of the flow path for at-line data acquisition and material analysis, wherein the recirculation loop connecting the flow cells to the flow path of the unit operation includes an external vessel, wherein the external vessel comprises a body in the form of a flexible or a rigid container defining an interior space, inlet and outlet ports, an optional impeller for fluid recirculation, and an optional probe or sensor, wherein the external vessel is a hold vessel, a release tank, a dilution tank, a mixing tank, or a stirred tank reactor.

22. The apparatus of claim 1, wherein the recirculation loop is connected via one or more valves to one or more of a material recycle flow path, a waste flow path, and a forward processing flow path.

23. The apparatus of claim 22, wherein the one or more valves is controlled by operation of the controller in accordance with one or more predetermined criteria.

24. The apparatus of claim 1, wherein the controller comprises an advanced process controller (APC).

25. The apparatus of claim 1, wherein the data acquisition and analysis layer includes multivariate data analysis, chemometric modeling and/or other advanced process modeling software.

26. The apparatus of claim 1, wherein the apparatus is fluidly coupled to at least one of the unit operations of the pharmaceutical or biopharmaceutical manufacturing process, wherein the apparatus is fluidly coupled in-line between the unit operation and a second unit operation in the process and the recirculation loop is connected via one or more valves to one or more of a material recycle flow path, a waste flow path, and a forward processing flow path.

27. The apparatus of claim 26, wherein the apparatus is fluidly coupled between the unit operation and a second unit operation in the process via an external vessel.

28. A method for real time release testing in a pharmaceutical or biopharmaceutical process, the method comprising connecting an apparatus according to claim 1 to a unit operation of the process to capture samples of the process fluid,
measuring an indication of at least one product attribute from the process fluid via operation of the one or more process analytical instruments of the process analytical technology hardware,
comparing the indication of the at least one product attribute against a predetermined range for the attribute via operation of a data acquisition and analysis software layer,
determining whether the indication is within or outside the predetermined range, and
executing a first set of instructions via the controller to release a volume of process fluid to a second unit operation in the process if the received indication is within the predetermined range,
executing a second set of instructions via the controller to release the volume of process fluid to either a recycle flow path or a waste flow path if the indication is outside the predetermined range, or
executing a third set of instructions via the controller to maintain the volume of process fluid in the recirculation loop until the indication is within the predetermined range.

29. The method of claim 28, comprising recirculating the volume of process fluid into an internal vessel of the housing via the sample port and one or more probes or sensors of one or more of the process analytical instruments of the process analytical technology hardware.

30. The method of claim 29, comprising capturing a sample of the process fluid from the sample port of the internal vessel or a sample port connected to a tubeset for offline analysis, online analysis, at-line analysis, in-line analysis, or a combination thereof, via automated sampling.
